(19) United States
(12) Patent Application Publication
(10) Pub. No.: US 2016/0058735 A1
(43) Pub. Date: Mar. 3, 2016

(54) METHODS OF TREATING HYPERHIDROSIS

(71) Applicant: Dermira, Inc., Menlo Park, CA (US)

(72) Inventors: Luis C. Pena, Menlo Park, CA (US); Dana B. DiBenedetti, Research Triangle Park, NC (US); Sheri E. Fehnel, Research Triangle Park, NC (US)

(21) Appl. No.: 14/837,446

(22) Filed: Aug. 27, 2015

Related U.S. Application Data
(60) Provisional application No. 62/042,660, filed on Aug. 27, 2014.
(60) Provisional application No. 62/171,041, filed on Jun. 4, 2015.

(51) Int. Cl. A61K 31/40; A61B 5/00; A61Q 15/00; A61K 8/49; A61K 9/00; A61K 31/185

(52) U.S. Cl. CPC A61K 31/40; A61K 9/0014; A61K 31/185; A61Q 15/00; A61K 8/4913; A61B 5/4266; A61K 2800/74

(57) ABSTRACT

Provided herein are methods of treating hyperhidrosis by administering an effective hyperhidrosis treatment and scoring hyperhidrosis in a subject in need thereof. In some embodiments, the treatment is a glycopyrronium compound and the scoring is an assessment provided herein, or a portion thereof.

### CROSS-REFERENCE TO RELATED APPLICATION

This application claims benefit of, and priority to under 35 U.S.C. §119(e), U.S. Provisional Application Ser. No. 62/042,660, entitled “METHOD OF TREATING HYPERHIDROSIS,” filed on Aug. 27, 2014, and U.S. Provisional Application Ser. No. 62/171,041, entitled “METHOD OF TREATING HYPERHIDROSIS,” filed on Jun. 4, 2015, which are incorporated by reference herein in their entirety.

### FIELD

Provided herein are methods of treating hyperhidrosis by administering a pharmaceutical composition comprising an agent effective for the treatment of hyperhidrosis and an assessment described herein.

### BACKGROUND

Hyperhidrosis is a condition of excessive sweating beyond what is physiologically required to maintain normal thermal regulation. Sweat is produced by glands in the skin and released to the skin surface through ducts. Sweat gland activity is controlled by the nervous system. The nervous system transmits signals to the sweat glands through the neurotransmitter acetylcholine. Primary hyperhidrosis, which is excessive sweating without a known cause, is localized and characteristically symmetric. It can affect the underarms, palms of the hands, soles of the feet, face (including neck and scalp), backs of the knees, trunk, groin, and other areas of the body. Several studies have demonstrated that excessive sweating often impedes normal daily activities and can result in occupational, emotional, psychological, social, and physical impairment.

In the United States, based on the most recent data available, the prevalence of hyperhidrosis is estimated to be about 2.8%. See Strutton et al., J. Am. Acad. Dermatol., 2005, 51:241-248. Approximately half of subjects afflicted with hyperhidrosis have axillary (underarm) hyperhidrosis.

A variety of treatments are available for hyperhidrosis, including antiperspirants containing metal salts (e.g., aluminum chloride), injection of botulinum toxin (Botox®), treatment with microwave heating devices, iontophoresis, surgical removal of sweat glands, and systemic or local treatment with anticholinergic compounds.

Treatment of hyperhidrosis may also involve the administration of one or more assessments, to evaluate the degree of hyperhidrosis and the effect of treatment on hyperhidrosis. However, current assessments are either limited in their ability to assess a wide range of symptoms of hyperhidrosis, or rely on general quality of life measures that are not specific to the treatment of hyperhidrosis. For example, the Hyperhidrosis Disease Severity Scale (HDSS) utilizes a single question with a score of 1 to 4 to assess hyperhidrosis. It is therefore limited in its ability to assess a wide range of symptoms of hyperhidrosis. See Solish et al., Dermatol. Surg., 2007, 33:908-923. On the other hand, a general quality-of-life questionnaire has been used to evaluate the surgical treatment of hyperhidrosis. See Ribas et al., Ann. Thorac. Surg., 2003, 76:886-891. This approach is not specific to the treatment of hyperhidrosis.

There is therefore a need for methods for the treatment of hyperhidrosis that integrate assessments that assess a wide range of symptoms that are specific to hyperhidrosis. The methods of treatment provided herein address this need.

### SUMMARY

Provided herein are methods of evaluating hyperhidrosis in a subject in need thereof, comprising administering an assessment provided herein, or a portion thereof, to the subject. In some embodiments, the assessment, or portion thereof, is a patient-reported outcome (PRO).

Also provided are methods of treating hyperhidrosis in a subject in need thereof, comprising administering an assessment provided herein, or a portion thereof, to the subject, and administering an effective hyperhidrosis treatment to the subject. In some embodiments, the effective hyperhidrosis treatment is selected from an anticholinergic agent, an antiperspirant, botulinum toxin, microwave heating, iontophoresis, and surgical removal of sweat glands. In some embodiments, the effective hyperhidrosis treatment comprises administration of a pharmaceutical composition that comprises a glycopyrronium compound.

Also provided are methods of treating hyperhidrosis in a subject in need thereof, comprising administering an assessment provided herein, or a portion thereof, to the subject, and administering a pharmaceutical composition comprising a therapeutically effective amount of an agent effective to treat hyperhidrosis to the subject. In some embodiments, the pharmaceutical composition comprises a glycopyrronium compound.

In some embodiments, provided herein is a method for treating hyperhidrosis in a subject comprising: (i) scoring the subject's sweating, during the past 24-hours, at its worst, on a scale of 0 to 10, at a value of 6 to 10 in a first scoring; and (ii) administering an effective hyperhidrosis treatment to the subject such that after treatment, and at least 24-hours after the first scoring, the subject's sweating is scored at a value of 0 to 5 in a second scoring. In some aspects, the sweating is underarm sweating.

In some embodiments, provided herein is a method for treating hyperhidrosis in a subject comprising: (i) scoring the impact of the subject's sweating on their activities during the past 24-hours as “an extreme amount,” “a great deal,” or “a moderate amount” in a first scoring; and (ii) administering an effective hyperhidrosis treatment to the subject such that after treatment, and at least 24-hours after the first scoring, the impact is “not at all” or “a little bit” in a second scoring. In some aspects, the sweating is underarm sweating.

In some embodiments, provided herein is a method for treating hyperhidrosis in a subject comprising: (i) scoring how bothered the subject was by their sweating, during the past 24-hours, as “extremely bothered,” “very bothered,” or “moderately bothered” in a first scoring; and (ii) administering an effective hyperhidrosis treatment to the subject, such that after treatment, and at least 24-hours after the first scoring, the subject is “not at all bothered” or “a little bothered” in a second scoring. In some aspects, the sweating is underarm sweating.

In some embodiments, provided herein is a method for treating hyperhidrosis in a subject comprising: (i) scoring whether the subject exhibited 6 symptoms of hyperhidrosis during the past 7 days at a value of 4 to 6 symptoms exhibited out of the 6 symptoms in a first scoring; and (ii) administering an effective hyperhidrosis treatment to the subject such that after treatment, and at least 7 days after the first scoring, the subject exhibits 0 to 3 symptoms in a second scoring. In some aspects, the hyperhidrosis is underarm hyperhidrosis.

In some embodiments, provided herein is a method for treating hyperhidrosis in a subject comprising: (i) administering an effective hyperhidrosis treatment to the subject; and (ii) scoring the subject's overall sweating at present, as compared to before treatment, as “much better,” “moderately better,” or “a little better” in a first scoring. In some aspects, the sweating is underarm sweating.

In some aspects, the effective hyperhidrosis treatment is selected from an anticholinergic agent, an antiperspirant (e.g., a metal salt such as aluminum chloride), a toxin (e.g., botulinum toxin), microwave heating (e.g., miraDry®), iontophoresis, surgical removal of sweat glands (e.g., excision, curettage, and/or liposuction), surgical destruction of nerves that transmit activating signals to sweat glands (i.e., sympathectomy), an ultrasound treatment, and a laser treatment.

In some aspects, wherein the effective hyperhidrosis treatment is an anticholinergic agent. In some aspects, the anticholinergic agent is applied topically. In some aspects, the anticholinergic agent is a glycopyrronium compound. In some aspects, the glycopyrronium compound is administered topically or orally. In some aspects, the glycopyrronium compound is administered topically, by a pharmaceutical composition comprising about 0.25% to about 6% (w/w) of the glycopyrronium compound. In some aspects, the glycopyrronium compound is selected from threo-glycopyrronium tosylate and threo-glycopyrronium bromide. In some aspects, the pharmaceutical composition is in the form of a pad or a wipe.

Also provided is a kit comprising: (i) an agent effective for the treatment of hyperhidrosis; and (ii) an assessment, wherein the assessment comprises one or more of: a Sweating Daily Diary, or a portion thereof; a Weekly Impact Items questionnaire; and a Patient Global Impression of Change Item.

Also provided are methods for treating selected patient populations experiencing hyperhidrosis. In some aspects, the patient population is selected based on the site of hyperhidrosis. In some aspects, the patient population is selected based on age. In some aspects, the patient population is selected based on gender. In some aspects, the patient population is selected based on a familial history of hyperhidrosis.

In some embodiments, the methods of treatment provided herein are used in concert with physiological assessments of hyperhidrosis. In some aspects, the physiological assessment of hyperhidrosis is selected from gravimetry, vapometry, electrical measurement, visual measurement, and combinations thereof.

In some embodiments, two or more of the answers to questions on an assessment provided herein are aggregated using a scoring algorithm.

The methods provided herein enable improved quantification of hyperhidrosis during and after treatment. The methods allow adjustment of the dose of the pharmaceutical composition to achieve effective therapy of hyperhidrosis, while minimizing undesirable side effects.

### BRIEF DESCRIPTION OF THE DRAWINGS

FIG. 1 provides efficacy data for 18 patients in whom the efficacy of Formulation A was assessed, as described in Example 4.1.

FIG. 2. provides a summary of the impact of the reference agent on disease severity, assessed as the proportion of patients achieving an improvement of at least two points in HDSS score from baseline to the end of the four-week treatment period, as described in Example 4.2.1. Intention to treat (ITT) population shown=all randomized patients dispensed study product (n=198). Patients with missing data points were considered non-responders.

FIG. 3 provides a summary of the impact of the reference agent on sweat production, assessed as the average absolute change in sweat production from baseline to the end of the four-week treatment period, as described in Example 4.2.1. ITT population shown=all randomized patients dispensed study product (n=198). The last available on-treatment observation was used to estimate missing data points.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures known in the art that are described or referenced herein are generally well understood and commonly employed using conventional methodologies by those skilled in the art.

As used herein, the singular forms “a,” “an,” and “the” include the plural referents unless the context clearly indicates otherwise.

As used herein, the term “about” refers to the stated value plus or minus 10%. For example, a value of “about 10” encompasses a range of 9 to 11.

As used herein, “glycopyrronium compound” means a compound of the formula:

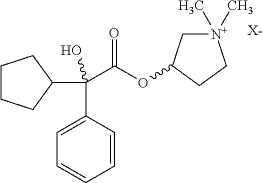

wherein X⁻ is a pharmaceutically acceptable counter ion that ionically associates with the glycopyrronium base.

As used herein, the phrase “pharmaceutically acceptable counter ion” refers to ions which retain the biological effectiveness and properties of the glycopyrronium base, which are not biologically or otherwise undesirable, and which carry an anionic charge. The glycopyrronium base forms salts by virtue of the presence of the quaternary ammonium thereon. The pharmaceutically acceptable counter ion may be prepared from inorganic or organic acids. Salts derived from inorganic acids include, but are not limited to, hydrochloric acid, hydrobromic acid, hydrogen fluoride, hydrogen iodide, sulfuric acid, nitric acid, phosphoric acid, and the like. Salts derived from organic acids include, but are not limited to, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. In some embodiments, the salt is derived from p-toluene sulfonic acid or hydrobromic acid.

In some embodiments, the glycopyrronium compound is glycopyrronium tosylate. In some embodiments, the glycopyrronium compound is glycopyrronium bromide.

The glycopyrronium base has two stereocenters, as indicated above. As such, also provided herein are methods of treatment with glycopyrronium compounds comprising one or more stereoisomers of glycopyrronium base, or a selected mixture of stereoisomers of glycopyrronium base, useful for the treatment of hyperhidrosis.

In some embodiments, the glycopyrronium compound is threo-glycopyrronium tosylate. In some embodiments, the glycopyrronium compound is threo-glycopyrronium bromide.

In some embodiments, the glycopyrronium compound comprises threo-glycopyrronium tosylate. In some embodiments, the glycopyrronium compound comprises threo-glycopyrronium bromide.

In some embodiments, the glycopyrronium compound comprises threo-glycopyrronium and erythro-glycopyrronium, wherein the threo-glycopyrronium is at least 95% of the total glycopyrronium content of the composition and the erythro-glycopyrronium is less than 5% of the total glycopyrronium content of the composition.

The phrase “glycopyrronium compound” also includes analogs of glycopyrronium capable of inhibiting hyperhidrosis wherein the chemical structure has been modified so as to introduce, modify and/or remove one or more functionalities of the structure. For example, such modification can result in the removal of a hydroxyl functionality, the introduction of an amine functionality, the introduction of a halo functionality, and the like. Insofar as the glycopyrronium analogues are capable of inhibiting hyperhidrosis they are encompassed by the definition of “glycopyrronium compound.”

As used herein, “scoring” refers, in certain embodiments, to administering an assessment provided herein, or a portion thereof, to subject, wherein the assessment is useful to evaluate hyperhidrosis. Generally, administering an assessment provided herein involves assigning a value to a symptom based on a scale, or from a selection of results or answers. For example, in some embodiments, scoring involves assigning a value from 0 to 10 that corresponds to the degree of underarm sweating during the past 24 hours, at its worst. See e.g., Question 2 of the Axillary Sweating Daily Diary provided in Example 1 of this disclosure. In other embodiments, scoring involves assigning a descriptive answer that quantifies the extent to which underarm sweating impacted daily activities during the past 24 hours. See e.g., Question 3 of the Axillary Sweating Daily Diary provided in Example 1 of this disclosure. Scoring can be performed by a patient (i.e., a patient-reported outcome) or by another person, such as a healthcare practitioner.

As used herein, “treating” or “treatment” of hyperhidrosis refers, in certain embodiments, to ameliorating hyperhidrosis that exists in a subject. In some embodiments, “treating” or “treatment” includes ameliorating at least one physical parameter of hyperhidrosis, such as sweating. In some embodiments, “treating” or “treatment” includes modulating the hyperhidrosis. In some embodiments, “treating” or “treatment” includes delaying or preventing the onset of hyperhidrosis (e.g., a prophylactic treatment). In some embodiments, “treating” or “treatment” includes mitigating the incidence of episodes of hyperhidrosis, by periodic administration of a pharmaceutical composition according to the methods provided herein.

As used herein, the term “therapeutically effective amount” or “effective amount” refers to an amount of a composition provided herein that is useful for treating hyperhidrosis.

As used herein, the term “effective hyperhidrosis treatment” includes any treatment that may be used in treating hyperhidrosis. Any suitable effective hyperhidrosis treatment may be used in the methods provided herein. Illustrative suitable effective hyperhidrosis treatments include, for example, agents for the treatment of hyperhidrosis as described elsewhere in this disclosure (e.g., anticholinergic agents, metal salts, or toxins), microwave heating, iontophoresis, surgical removal of sweat glands, sympathectomy, ultrasound, and laser-based treatment. In particular embodiments, agent is a glycopyrronium compound.

As used herein, the terms “subject” and “patient” mean a mammalian subject. Exemplary subjects include, but are not limited to humans, monkeys, cows, horses, camels, goats and sheep. In certain embodiments, the subject is a human.

### 2. Overview of Methods of Evaluating and Treating Hyperhidrosis

In some embodiments, provided herein is a method for treating hyperhidrosis in a subject comprising: (i) scoring the subject's underarm sweating, during the past 24-hours, at its worst, on a scale of 0 to 10, at a value of 6 to 10 in a first scoring; and (ii) administering a sufficient amount of a topical pharmaceutical composition comprising about 0.25% to about 6% (w/w) of a glycopyrronium compound to the subject such that after treatment with the pharmaceutical composition, and at least 24-hours after the first scoring, the subject's underarm sweating is scored at a value of 0 to 5 in a second scoring. In some aspects, 0 in the scale corresponds to “no sweating at all,” while 10 in the scale corresponds to “worst possible sweating,” with each integer between 0 and 10 corresponding to increased sweating as the integers increase.

In some embodiments, provided herein is a method for treating hyperhidrosis in a subject comprising: (i) scoring the impact of the subject's underarm sweating on their activities during the past 24-hours as “an extreme amount,” “a great deal,” or “a moderate amount” in a first scoring; and (ii) administering a sufficient amount of a topical pharmaceutical composition comprising about 0.25% to about 6% (w/w) of a glycopyrronium compound to the subject such that after treatment with the pharmaceutical composition, and at least 24-hours after the first scoring, the impact is “not at all” or “a little bit” in a second scoring.

In some embodiments, provided herein is a method for treating hyperhidrosis in a subject comprising: (i) scoring how bothered the subject was by their underarm sweating, during the past 24-hours, as “extremely bothered,” “very bothered,” or “moderately bothered” in a first scoring; and (ii) administering a sufficient amount of a topical pharmaceutical composition comprising about 0.25% to about 6% (w/w) of a glycopyrronium compound to the subject, such that after treatment with the pharmaceutical composition, and at least 24-hours after the first scoring, the subject is “not at all bothered” or “a little bothered” in a second scoring.

In some embodiments, provided herein is a method for treating hyperhidrosis in a subject comprising: (i) scoring whether the subject exhibited 6 symptoms of underarm hyperhidrosis during the past 7 days at a value of 4 to 6 symptoms exhibited out of the 6 symptoms in a first scoring; and (ii) administering a sufficient amount of a topical pharmaceutical composition comprising about 0.25% to about 6% (w/w) of a glycopyrronium compound to the subject such that after treatment with the pharmaceutical composition, and at least 7 days after the first scoring, the subject exhibits 0 to 3 symptoms in a second scoring.

In some embodiments, provided herein is a method for treating hyperhidrosis in a subject comprising: (i) administering a topical pharmaceutical composition comprising about 0.25% to about 6% (w/w) of a glycopyrronium compound to the subject; and (ii) scoring the subject's overall underarm sweating at present, as compared to before treatment, as “much better,” “moderately better,” or “a little better” in a first scoring.

In some embodiments, provided herein is a method for treating hyperhidrosis in a subject comprising: (i) scoring the subject's underarm sweating, during the past 24-hours, at its worst, on a scale of 0 to 10, at a value of 6 to 10 in a first scoring; and (ii) administering a sufficient amount of a topical pharmaceutical composition comprising about 0.25% to about 6% (w/w) of a glycopyrronium compound to the subject such that after treatment with the pharmaceutical composition, and at least 24-hours after the first scoring, the subject's underarm sweating is scored at a value of 0 to 5 in a second scoring.

In some aspects, the method further comprises continuing to administer a sufficient amount of the pharmaceutical composition to the subject, such that in a third scoring, at least 24-hours after the second scoring, the subject's underarm sweating is scored at a value of 0 to 5.

In some aspects, the value in the first scoring is 7 to 10, and the value in the second scoring is 0 to 6.

In some aspects, the method further comprises adjusting the amount of the pharmaceutical composition administered to the subject based on the value in the second scoring.

In some aspects, one or more of the first, second, and third scoring steps comprises administering a questionnaire to the subject. In some aspects, the questionnaire comprises a question asking the subject to rate their underarm sweating, during the past 24-hours, at its worst, on a scale of 0 to 10, in increments of 1.

In some embodiments, provided herein is a method for treating hyperhidrosis in a subject comprising: (i) scoring the impact of the subject's underarm sweating on their activities during the past 24-hours as “an extreme amount,” “a great deal,” or “a moderate amount” in a first scoring; and (ii) administering a sufficient amount of a topical pharmaceutical composition comprising about 0.25% to about 6% (w/w) of a glycopyrronium compound to the subject such that after treatment with the pharmaceutical composition, and at least 24-hours after the first scoring, the impact is “not at all” or “a little bit” in a second scoring.

In some aspects, the method further comprises continuing to administer a sufficient amount of the pharmaceutical composition to the subject, such that in a third scoring, at least 24-hours after the second scoring, the impact is “not at all” or “a little bit.”

In some aspects, the impact in the first scoring is “an extreme amount” or “a great deal,” and the impact in the second scoring is “a moderate amount,” “a little bit,” or “not at all.”

In some aspects, the method further comprises adjusting the amount of the pharmaceutical composition administered to the subject based on the second scoring.

In some aspects, one or more of the first, second, and third scoring steps comprises administering a questionnaire to the subject. In some aspects, the questionnaire comprises a question asking the subject to what extent their underarm sweating impacted their activities during the past 24-hours, on a scale of “not at all,” “a little bit,” “a moderate amount,” “a great deal,” and “an extreme amount.”

In some embodiments, provided herein is a method for treating hyperhidrosis in a subject comprising: (i) scoring how bothered the subject was by their underarm sweating, during the past 24-hours, as “extremely bothered,” “very bothered,” or “moderately bothered” in a first scoring; and (ii) administering a sufficient amount of a topical pharmaceutical composition comprising about 0.25% to about 6% (w/w) of a glycopyrronium compound to the subject, such that after treatment with the pharmaceutical composition, and at least 24-hours after the first scoring, the subject is “not at all bothered” or “a little bothered” in a second scoring.

In some aspects, the method further comprises continuing to administer a sufficient amount of the pharmaceutical composition to the subject, such that in a third scoring, at least 24-hours after the second scoring, the subject is “not at all bothered” or “a little bothered.”

In some aspects, the subject is “extremely bothered” or “very bothered” in the first scoring and “moderately bothered,” “a little bothered,” or “not at all bothered” in the second scoring.

In some aspects, the method further comprises adjusting the amount of the pharmaceutical composition administered to the subject based on the second scoring.

In some aspects, one or more of the first, second, and third scoring steps comprises administering a questionnaire to the subject. In some aspects, the questionnaire comprises a question asking the subject how bothered they were by their underarm sweating during the past 24-hours, on a scale of “not at all bothered,” “a little bothered,” “moderately bothered,” “very bothered,” and “extremely bothered.”

In some embodiments, provided herein is a method for treating hyperhidrosis in a subject comprising: (i) scoring whether the subject exhibited 6 symptoms of underarm hyperhidrosis during the past 7 days at a value of 4 to 6 symptoms exhibited out of the 6 symptoms in a first scoring; and (ii) administering a sufficient amount of a topical pharmaceutical composition comprising about 0.25% to about 6% (w/w) of a glycopyrronium compound to the subject such that after treatment with the pharmaceutical composition, and at least 7 days after the first scoring, the subject exhibits 0 to 3 symptoms in a second scoring.

In some aspects, the method further comprises continuing to administer a sufficient amount of the pharmaceutical composition to the subject, such that in a third scoring, at least 7 days after the second scoring, the subject exhibits 0 to 3 symptoms.

In some aspects, the subject exhibits 5 to 6 symptoms in the first scoring and 0 to 4 symptoms in the second scoring.

In some aspects, the method further comprises adjusting the amount of the pharmaceutical composition administered to the subject based on the symptoms exhibited in the second scoring.

In some aspects, one or more of the first, second and third scoring steps comprises administering a questionnaire to the subject. In some aspects, the questionnaire comprises a question asking the subject to respond with “yes” or “no” to the following questions: (a) “During the past 7 days, did you ever have to change your shirt during the day because of your underarm sweating?”; (b) “During the past 7 days, did you ever have to take more than 1 shower or bath a day because of your underarm sweating?”; (c) “During the past 7 days, did you ever feel less confident in yourself because of your underarm sweating?”; (d) “During the past 7 days, did you ever feel embarrassed by your underarm sweating?”; (e) “During the past 7 days, did you ever avoid interactions with other people because of your underarm sweating?”; and (f) “During the past 7 days, did your underarm sweating ever keep you from doing an activity you wanted or needed to do?”

In some embodiments, provided herein is a method for treating hyperhidrosis in a subject comprising: (i) administering a topical pharmaceutical composition comprising about 0.25% to about 6% (w/w) of a glycopyrronium compound to the subject; and (ii) scoring the subject's overall underarm sweating at present, as compared to before treatment, as “much better,” “moderately better,” or “a little better” in a first scoring.

In some aspects, the method further comprises continuing to administer a sufficient amount of the pharmaceutical composition to the subject, such that in a second scoring, at least seven days after the first scoring, the subject's overall underarm sweating, as compared to before treatment, is scored as “much better,” “moderately better,” or “a little better.”

In some aspects, the subject's overall underarm sweating in the first scoring is scored as “much better” or “moderately better.”

In some aspects, the method further comprises adjusting the amount of the pharmaceutical composition administered to the subject based on the first scoring.

In some aspects, one or more of the first or second scoring steps comprises administering a questionnaire to the subject. In some aspects, the questionnaire comprises a question asking the subject to rate their underarm sweating now as compared to before starting the study treatment, on a scale of “much better,” “moderately better,” “a little better,” “no difference,” “a little worse,” “moderately worse,” and “much worse.”

In some embodiments, provided herein is a method for treating hyperhidrosis in a subject comprising: (a) administering a first questionnaire to the subject before treatment with a topical pharmaceutical composition comprising about 0.25% to about 6% (w/w) of a glycopyrronium compound, wherein the first questionnaire comprises a question asking the subject to rate their underarm sweating, during the past 24-hours, at its worst, on a scale of 0 to 10, and wherein the subject rates their underarm sweating on the first questionnaire as 6 to 10; and (b) administering a sufficient amount of the pharmaceutical composition to the subject such that on administering a second questionnaire to the subject after treatment with the pharmaceutical composition, and at least 24-hours after administration of the first questionnaire, wherein the second questionnaire comprises a question asking the subject to rate their underarm sweating, during the past 24-hours, at its worst, on a scale of 0 to 10, the subject rates their underarm sweating on the second questionnaire as 0 to 5.

In some embodiments, provided herein is a method for treating hyperhidrosis in a subject comprising: (a) administering a first questionnaire to the subject before treatment with a topical pharmaceutical composition comprising about 0.25% to about 6% (w/w) of a glycopyrronium compound, wherein the first questionnaire comprises a question asking the subject to what extent their underarm sweating impacted their activities during the past 24-hours, wherein the subject rates the impact on the first questionnaire as “an extreme amount,” “a great deal,” or “a moderate amount;” and (b) administering a sufficient amount of the pharmaceutical composition to the subject such that on administering a second questionnaire to the subject after treatment with the pharmaceutical composition, and at least 24-hours after administration of the first questionnaire, wherein the second questionnaire comprises a question asking the subject to what extent their underarm sweating impacted their activities during the past 24-hours, the subject rates the impact on the second questionnaire as “not at all” or “a little bit.”

In some embodiments, provided herein is a method for treating hyperhidrosis in a subject comprising: (a) administering a first questionnaire to the subject before treatment with a topical pharmaceutical composition comprising about 0.25% to about 6% (w/w) of a glycopyrronium compound, wherein the first questionnaire comprises a question asking the subject how bothered they were by their underarm sweating, during the past 24-hours, wherein the subject reports that they were “extremely bothered,” “very bothered,” or “moderately bothered” in the first questionnaire; and (b) administering a sufficient amount of the pharmaceutical composition to the subject such that on administering a second questionnaire to the subject after treatment with the pharmaceutical composition, and at least 24-hours after administration of the first questionnaire, wherein the second questionnaire comprises a question asking the subject how bothered they were by their underarm sweating, during the past 24-hours, the subject reports that they were “not at all bothered” or “a little bothered” on the second questionnaire.

In some embodiments, provided herein is a method for treating hyperhidrosis in a subject comprising: (a) administering a first questionnaire to the subject before treatment with a topical pharmaceutical composition comprising about 0.25% to about 6% (w/w) of a glycopyrronium compound, wherein the first questionnaire comprises a battery of six questions, each of the six questions asking whether the subject has or has not experienced a symptom of hyperhidrosis within the past seven days, and wherein the subject reports experiencing 4-6 of the symptoms on the first questionnaire; and (b) administering a sufficient amount of the pharmaceutical composition to the subject such that on administering a second questionnaire to the subject after treatment with the pharmaceutical composition, wherein the second questionnaire comprises the battery of six questions, and the subject reports experiencing 0-3 of the symptoms on the second questionnaire.

In some embodiments, provided herein is a method for treating hyperhidrosis in a subject comprising: (a) administering a topical pharmaceutical composition comprising about 0.25% to about 6% (w/w) of a glycopyrronium compound to the subject; and (b) administering a first questionnaire to the subject after treatment with the pharmaceutical composition, wherein the first questionnaire comprises a question asking the subject to rate their overall underarm sweating at present, as compared to before treatment, and wherein the subject rates their overall underarm sweating at present, as compared to before treatment, as “much better,” “moderately better,” or “a little better.”

In some embodiments, certain aspects of these methods, such as one or more of the active agent and dose thereof, the nature of the questionnaire, the response on the questionnaire, the number of times the questionnaire is administered, and the frequency of administration of the questionnaire are varied according to the description provided herein.

In some embodiments, the amount of the pharmaceutical composition administered to the subject is adjusted based on the subject's response on one or more of the administered questionnaires.

In some embodiments, the questionnaire is selected from a written questionnaire, a verbal questionnaire, and an electronic questionnaire. In some aspects, the electronic questionnaire is provided via a website. In some embodiments, the electronic questionnaire is provided via a text message. In some embodiments, the electronic questionnaire is provided via email. In some embodiments, the electronic questionnaire is provided via a mobile application.

In some embodiments, the questionnaire is an electronic questionnaire, and a result from the electronic questionnaire is transmitted to a healthcare practitioner. In some embodiments, the healthcare practitioner is selected from a physician, a nurse, and a pharmacist. In some embodiments, the healthcare practitioner adjusts the amount of the pharmaceutical composition administered to the subject based on the subject's response(s) on one or more of the questionnaires, or portions thereof.

In some embodiments, the pharmaceutical composition is in the form of a pad or a wipe. In some embodiments, the pad or the wipe further comprises ethanol. In some embodiments, the pad or the wipe further comprises a hydrophilic polymer and a hydrophobic polymer. In some embodiments, the hydrophilic polymer is polyvinyl pyrrolidone (PVP) and the hydrophobic polymer is a butyl ester of polyvinylmethylether/maleic anhydride (PVM/MA) copolymer. In some embodiments, the pad or the wipe comprises a solution with a pH of about 3.5 to about 6.0.

Also provided are kits for practice of the methods provided herein. In some embodiments, a kit comprises a topical pharmaceutical composition comprising about 0.25% to about 6% (w/w) of a glycopyrronium compound; and an assessment, wherein the assessment comprises one or more of: a Sweating Daily Diary (or a portion thereof); a Weekly Impact Items questionnaire; and a Patient Global Impression of Change Item.

In some embodiments, the kit further comprises instructions for the administration of the assessment. In some embodiments, the kit further comprises instructions for the administration of the topical pharmaceutical composition, based on the results of the assessment.

### 3. Methods of Evaluating and Treating Hyperhidrosis

Provided herein are methods of evaluating hyperhidrosis in a subject in need thereof, comprising administering an assessment provided herein, or a portion thereof, to the subject.

Also provided are methods of treating hyperhidrosis in a subject in need thereof, comprising administering an assessment provided herein, or a portion thereof, to the subject, and administering a pharmaceutical composition comprising a therapeutically effective amount of an agent effective to treat hyperhidrosis to the subject. In some embodiments, the pharmaceutical composition comprises a glycopyrronium compound.

The assessment may be provided to the patient in any suitable form. In some embodiments, the assessment is provided to the patient in the form of a questionnaire. In some aspects, the questionnaire is provided to the patient in a printed form. In some embodiments, the questionnaire is provided to the patient in an electronic form. In some embodiments, the questionnaire is provided to the patient in a verbal form, for example, during a consultation with a healthcare practitioner.

In some embodiments, the hyperhidrosis is primary hyperhidrosis. The term “primary hyperhidrosis” (also known as “focal hyperhidrosis”), as used herein, generally refers to localized excessive sweating that is not caused by another medical condition, and that is not a side effect of medications.

The methods provided herein may be used to treat any suitable form of primary hyperhidrosis. Illustrative forms of primary hyperhidrosis include, for example, hyperhidrosis of the underarms, palms of the hands, soles of the feet, face (including neck and scalp), backs of the knees, trunk, groin, and other areas of the body.

### 3.1. Methods of Evaluation and Treatment Comprising Administering an Axillary Sweating Daily Diary (ASDD), a Weekly Impact Items Questionnaire, and/or a Patient Global Impression of Change Item

In some embodiments, the assessment is the Axillary Sweating Daily Diary (ASDD), designed to measure the severity and impact of underarm sweating during the preceding 24-hour period. Example 1 provides the ASDD.

The ASDD may be administered once or more than once. If administered more than once, the ASDD may be administered according to any suitable schedule. In some embodiments, the ASDD is administered every 24-hours. In some embodiments, the ASDD is administered every 48-hours. In some embodiments, the ASDD is administered every 72-hours. In some embodiments, the ASDD is administered every 96-hours. In some embodiments, the ASDD is administered every 120-hours. In some embodiments, the ASDD is administered every 96-hours. In some embodiments, the ASDD is administered every 144-hours. In some embodiments, the ASDD is administered every 96-hours. In some embodiments, the ASDD is administered every 168-hours.

In some embodiments, the assessment is the Weekly Impact Items questionnaire, designed to measure the severity and impact of underarm sweating during the preceding seven day period. Example 2 provides the Weekly Impact Items questionnaire.

The Weekly Impact Items questionnaire may be administered once or more than once. If administered more than once, the Weekly Impact Items questionnaire may be administered according to any suitable schedule. In some embodiments, the Weekly Impact Items questionnaire is administered every 7 days. In some embodiments, the Weekly Impact Items questionnaire is administered every 14 days. In some embodiments, the Weekly Impact Items questionnaire is administered every 21 days. In some embodiments, the Weekly Impact Items questionnaire is administered every 28 days. In some embodiments, the Weekly Impact Items questionnaire is administered monthly.

In some embodiments, the assessment is the Patient Global Impression of Change Item, designed to measure underarm sweating at present, as compared to before beginning therapy. Example 3 provides the Patient Global Impression of Change Item.

The Patient Global Impression of Change Item may be administered once or more than once. If administered more than once, the Patient Global Impression of Change Item may be administered according to any suitable schedule. In some embodiments, the Patient Global Impression of Change Item is administered every 7 days. In some embodiments, the Patient Global Impression of Change Item is administered every 14 days. In some embodiments, the Patient Global Impression of Change Item is administered every 21 days. In some embodiments, the Patient Global Impression of Change Item is administered every 28 days. In some embodiments, the Patient Global Impression of Change Item is administered monthly. In some embodiments, the Patient Global Impression of Change Item is administered quarterly. In some embodiments, the Patient Global Impression of Change Item is administered semi-annually. In some embodiments, the Patient Global Impression of Change Item is administered yearly.

In some embodiments, more than one of these assessments is administered to the subject. In some aspects, two of the three assessments are administered to the subject. In some aspects, all three of the assessments are administered to the subject.

In some embodiments, a subset of the questions on the ASDD are administered to the subject. In some embodiments, the subset of questions comprises one or more of Question 2, Question 3, and Question 4 from the ASDD provided in Example 1.

In some embodiments, the dose of the agent effective for the treatment of hyperhidrosis is adjusted based on the results of one or more of the questionnaires provided herein. In some embodiments, the dose is increased based on the results of one or more questionnaires provided herein. In some embodiments, the dose is decreased based on the results of one or more questionnaires provided herein.

### 3.1.1. Other Sweating Daily Diaries (SDDs), Weekly Impact Items, and Patient Global Impression of Change Items

The ASDD, Weekly Impact Items, and Patient Global Impression of Change Item provided in Examples 1-3 are customized to evaluate underarm sweating, and are provided for illustrative purposes. However, one of skill in the art will recognize that these assessments may also be customized to measure any other suitable form of primary hyperhidrosis, for example, by substituting the word “underarm” in the assessments with the appropriate region of the body. Illustrative appropriate regions of the body include, for example, the palms of the hands, the soles of the feet, the face (including the neck and the scalp), the backs of the knees, the trunk, and the groin. These assessments customized to measure other forms of primary hyperhidrosis may also be administered according the schedules provided above, or according to any other suitable schedule. Similarly, subsections of these assessments (e.g., one or more of Questions 2, 3, and 4 from a customized SDD) may be administered to the subject.

### 3.2. Methods of Evaluation and Treatment Comprising Scoring Sweating During the Past 24-Hours, at Worst

In some embodiments, provided herein is a method for evaluating hyperhidrosis in a subject comprising scoring the subject's sweating during the past 24-hours, at its worst. In some embodiments, the scoring is on a scale from 0 to 10, in increments of one (i.e., 0, 1, 2, 3, 4, 5, 6, 7, 8, 9, and 10), where 0 represents no sweating at all, and 10 represents the worst possible sweating, or an equivalent rating scale. In some embodiments, the scoring comprises administering a questionnaire to the subject. See Question 2 in Example 1 of this disclosure.

In some embodiments, provided herein is a method for treating hyperhidrosis in a subject comprising: (i) scoring the subject's sweating, during the past 24-hours, at its worst, on a scale of 0 to 10, at a value of a₁ to b₁ in a first scoring; and (ii) administering a sufficient amount of a pharmaceutical composition comprising a therapeutically effective amount of an agent effective to treat hyperhidrosis to the subject such that after treatment with the pharmaceutical composition, the subject's sweating is scored at a value of a₂ to b₂ in a second scoring.

In some embodiments, the range a₁ to b₁ is selected from 5 to 10, 6 to 10, 7 to 10, 8 to 10, or 9 to 10.

In some embodiments, the range a₂ to b₂ is selected from 0 to 5, 0 to 4, 0 to 3, 0 to 2, or 0 to 1.

In some embodiments, a₁-a₂ is in the range of 5 to 9, 6 to 9, 7 to 9, or 8 to 9.

In some embodiments, b₁-b₂ is in the range of 5 to 9, 6 to 9, 7 to 9, or 8 to 9.

In some embodiments, a₁ to b₁ is 5 to 10 and a₂ to b₂ is selected from 0 to 4, 0 to 3, 0 to 2, or 0 to 1. In some embodiments, a₁ to b₁ is 6 to 10 and a₂ to b₂ is selected from 0 to 5, 0 to 4, 0 to 3, 0 to 2, or 0 to 1. In some embodiments, a₁ to b₁ is 7 to 10 and a₂ to b₂ is selected from 0 to 6, 0 to 5, 0 to 4, 0 to 3, 0 to 2, or 0 to 1. In some embodiments, a₁ to b₁ is 8 to 10 and a₂ to b₂ is selected from 0 to 7, 0 to 6, 0 to 5, 0 to 4, 0 to 3, 0 to 2, or 0 to 1. In some embodiments, a₁ to b₁ is 9 to 10 and a₂ to b₂ is selected from 0 to 8, 0 to 7, 0 to 6, 0 to 5, 0 to 4, 0 to 3, 0 to 2, or 0 to 1.

In some embodiments, the second scoring is performed at least 24-hours after the first scoring. In some aspects, the second scoring is performed at least 48-hours after the first scoring. In some aspects, the second scoring is performed at least 72-hours after the first scoring. In some aspects, the second scoring is performed at least 96-hours after the first scoring. In some aspects, the second scoring is performed at least 120-hours after the first scoring. In some aspects, the second scoring is performed at least 144-hours after the first scoring. In some aspects, the second scoring is performed at least 168-hours after the first scoring.

In some embodiments, a third scoring is performed, wherein the third scoring comprises scoring the subject's sweating, during the past 24-hours, at its worst, on a scale of 0 to 10, at a value of a₃ to b₃.

In some embodiments, the range a₃ to b₃ is selected from 0 to 5, 0 to 4, 0 to 3, 0 to 2, or 0 to 1.

In some embodiments, a₂-a₃ is in the range of 5 to 9, 6 to 9, 7 to 9, or 8 to 9.

In some embodiments, b₂-b₃ is in the range of 5 to 9, 6 to 9, 7 to 9, or 8 to 9.

In some embodiments, a₂ to b₂ is 5 to 10 and a₃ to b₃ is selected from 0 to 4, 0 to 3, 0 to 2, or 0 to 1. In some embodiments, a₂ to b₂ is 6 to 10 and a₃ to b₃ is selected from 0 to 5, 0 to 4, 0 to 3, 0 to 2, or 0 to 1. In some embodiments, a₂ to b₂ is 7 to 10 and a₃ to b₃ is selected from 0 to 6, 0 to 5, 0 to 4, 0 to 3, 0 to 2, or 0 to 1. In some embodiments, a₂ to b₂ is 8 to 10 and a₃ to b₃ is selected from 0 to 7, 0 to 6, 0 to 5, 0 to 4, 0 to 3, 0 to 2, or 0 to 1. In some embodiments, a₂ to b₂ is 9 to 10 and a₃ to b₃ is selected from 0 to 8, 0 to 7, 0 to 6, 0 to 5, 0 to 4, 0 to 3, 0 to 2, or 0 to 1.

In some embodiments, the third scoring is performed at least 24-hours after the second scoring. In some aspects, the third scoring is performed at least 48-hours after the second scoring. In some aspects, the third scoring is performed at least 72-hours after the second scoring. In some aspects, the third scoring is performed at least 96-hours after the second scoring. In some aspects, the third scoring is performed at least 120-hours after the second scoring. In some aspects, the third scoring is performed at least 144-hours after the second scoring. In some aspects, the third scoring is performed at least 168-hours after the second scoring.

In some embodiments, a further scoring is performed, wherein the further scoring comprises scoring the subject's sweating, during the past 24-hours, at its worst, on a scale of 0 to 10, at a value of aₓ to bₓ, where x is 4 or greater.

In some embodiments, the range aₓ to bₓ is selected from 0 to 5, 0 to 4, 0 to 3, 0 to 2, or 0 to 1.

In some embodiments, aₓ₋₁-aₓ is in the range of 5 to 9, 6 to 9, 7 to 9, or 8 to 9.

In some embodiments, bₓ₋₁-bₓ is in the range of 5 to 9, 6 to 9, 7 to 9, or 8 to 9.

In some embodiments, aₓ₋₁ to bₓ₋₁ is 5 to 10 and aₓ to bₓ is selected from 0 to 4, 0 to 3, 0 to 2, or 0 to 1. In some embodiments, aₓ₋₁ to bₓ₋₁ is 6 to 10 and aₓ to bₓ is selected from 0 to 5, 0 to 4, 0 to 3, 0 to 2, or 0 to 1. In some embodiments, (aₓ₋₁ to bₓ₋₁ is 7 to 10 and aₓ to bₓ is selected from 0 to 6, 0 to 5, 0 to 4, 0 to 3, 0 to 2, or 0 to 1. In some embodiments, (aₓ₋₁ to bₓ₋₁ is 8 to 10 and aₓ to bₓ is selected from 0 to 7, 0 to 6, 0 to 5, 0 to 4, 0 to 3, 0 to 2, or 0 to 1. In some embodiments, (aₓ₋₁ to bₓ₋₁ is 9 to 10 and aₓ to bₓ is selected from 0 to 8, 0 to 7, 0 to 6, 0 to 5, 0 to 4, 0 to 3, 0 to 2, or 0 to 1.

In some embodiments, the x scoring is performed at least 24-hours after the x-1 scoring. In some aspects, the x scoring is performed at least 48-hours after the x-1 scoring. In some aspects, the x scoring is performed at least 72-hours after the x-1 scoring. In some aspects, the x scoring is performed at least 96-hours after the x-1 scoring. In some aspects, the x scoring is performed at least 120-hours after the x-1 scoring. In some aspects, the x scoring is performed at least 144-hours after the x-1 scoring. In some aspects, the x scoring is performed at least 168-hours after the x-1 scoring.

In some embodiments, one or more of the scoring steps comprises administering a questionnaire to the subject.

In some embodiments, provided herein is a method for treating hyperhidrosis in a subject comprising: (i) administering a first questionnaire to the subject before treatment with a pharmaceutical composition comprising a therapeutically effective amount of an agent effective to treat hyperhidrosis, wherein the first questionnaire comprises a question asking the subject to rate their sweating, during the past 24-hours, at its worst, on a scale of 0 to 10, and wherein the subject rates their sweating on the first questionnaire as within the range c₁ to d₁; and (ii) administering a sufficient amount of the pharmaceutical composition to the subject such that on administering a second questionnaire to the subject after treatment with the pharmaceutical composition, wherein the second questionnaire comprises a question asking the subject to rate their sweating, during the past 24-hours, at its worst, on a scale of 0 to 10, the subject rates their sweating on the second questionnaire as within the range c₂ to d₂.

In some embodiments, the range c₁ to d₁ is selected from 5 to 10, 6 to 10, 7 to 10, 8 to 10, or 9 to 10.

In some embodiments, the range c₂ to d₂ is selected from 0 to 5, 0 to 4, 0 to 3, 0 to 2, or 0 to 1.

In some embodiments, c₁-c₂ is in the range of 5 to 9, 6 to 9, 7 to 9, or 8 to 9.

In some embodiments, d₁-d₂ is in the range of 5 to 9, 6 to 9, 7 to 9, or 8 to 9.

In some embodiments, c₁ to d₁ is 5 to 10 and c₂ to d₂ is selected from 0 to 4, 0 to 3, 0 to 2, or 0 to 1. In some embodiments, c₁ to d₁ is 6 to 10 and c₂ to d₂ is selected from 0 to 5, 0 to 4, 0 to 3, 0 to 2, or 0 to 1. In some embodiments, c₁ to d₁ is 7 to 10 and c₂ to d₂ is selected from 0 to 6, 0 to 5, 0 to 4, 0 to 3, 0 to 2, or 0 to 1. In some embodiments, c₁ to d₁ is 8 to 10 and c₂ to d₂ is selected from 0 to 7, 0 to 6, 0 to 5, 0 to 4, 0 to 3, 0 to 2, or 0 to 1. In some embodiments, c₁ to d₁ is 9 to 10 and c₂ to d₂ is selected from 0 to 8, 0 to 7, 0 to 6, 0 to 5, 0 to 4, 0 to 3, 0 to 2, or 0 to 1.

In some embodiments, the second questionnaire is administered at least 24-hours after the first questionnaire. In some aspects, the second questionnaire is administered at least 48-hours after the first questionnaire. In some aspects, the second questionnaire is administered at least 72-hours after the first questionnaire. In some aspects, the second questionnaire is administered at least 96-hours after the first questionnaire. In some aspects, the second questionnaire is administered at least 120-hours after the first questionnaire. In some aspects, the second questionnaire is administered at least 144-hours after the first questionnaire. In some aspects, the second questionnaire is administered at least 168-hours after the first questionnaire.

In some embodiments, a third questionnaire is administered to the subject, wherein the third questionnaire comprises a question asking the subject to rate their sweating, during the past 24-hours, at its worst, on a scale of 0 to 10, the subject rates their underarm sweating on the third questionnaire as within the range c₃ to d₃.

In some embodiments, the range c₃ to d₃ is selected from 0 to 5, 0 to 4, 0 to 3, 0 to 2, or 0 to 1.

In some embodiments, c₂-c₃ is in the range of 5 to 9, 6 to 9, 7 to 9, or 8 to 9.

In some embodiments, d₂-d₃ is in the range of 5 to 9, 6 to 9, 7 to 9, or 8 to 9.

In some embodiments, c₂ to d₂ is 5 to 10 and c₃ to d₃ is selected from 0 to 4, 0 to 3, 0 to 2, or 0 to 1. In some embodiments, c₂ to d₂ is 6 to 10 and c₃ to d₃ is selected from 0 to 5, 0 to 4, 0 to 3, 0 to 2, or 0 to 1. In some embodiments, c₂ to d₂ is 7 to 10 and c₃ to d₃ is selected from 0 to 6, 0 to 5, 0 to 4, 0 to 3, 0 to 2, or 0 to 1. In some embodiments, c₂ to d₂ is 8 to 10 and c₃ to d₃ is selected from 0 to 7, 0 to 6, 0 to 5, 0 to 4, 0 to 3, 0 to 2, or 0 to 1. In some embodiments, c₂ to d₂ is 9 to 10 and c₃ to d₃ is selected from 0 to 8, 0 to 7, 0 to 6, 0 to 5, 0 to 4, 0 to 3, 0 to 2, or 0 to 1.

In some embodiments, the third questionnaire is administered at least 24-hours after the second questionnaire. In some aspects, the third questionnaire is administered at least 48-hours after the second questionnaire. In some aspects, the third questionnaire is administered at least 72-hours after the second questionnaire. In some aspects, the third questionnaire is administered at least 96-hours after the second questionnaire. In some aspects, the third questionnaire is administered at least 120-hours after the second questionnaire. In some aspects, the third questionnaire is administered at least 144-hours after the second questionnaire. In some aspects, the third questionnaire is administered at least 168-hours after the second questionnaire.

In some embodiments, a further questionnaire is administered to the subject, wherein the further questionnaire comprises a question asking the subject to rate their sweating, during the past 24-hours, at its worst, on a scale of 0 to 10, the subject rates their underarm sweating on the further questionnaire as within the range cₓ to dₓ, where x is 4 or greater.

In some embodiments, the range cₓ to dₓ is selected from 0 to 5, 0 to 4, 0 to 3, 0 to 2, or 0 to 1.

In some embodiments, cₓ₋₁-dₓ is in the range of 5 to 9, 6 to 9, 7 to 9, or 8 to 9.

In some embodiments, cₓ₋₁-dₓ is in the range of 5 to 9, 6 to 9, 7 to 9, or 8 to 9.

In some embodiments, cₓ₋₁ to dₓ₋₁ is 5 to 10 and cₓ to dₓ is selected from 0 to 4, 0 to 3, 0 to 2, or 0 to 1. In some embodiments, cₓ₋₁ to dₓ₋₁ is 6 to 10 and cₓ to dₓ is selected from 0 to 5, 0 to 4, 0 to 3, 0 to 2, or 0 to 1. In some embodiments, cₓ₋₁ to dₓ₋₁ is 7 to 10 and cₓ to dₓ is selected from 0 to 6, 0 to 5, 0 to 4, 0 to 3, 0 to 2, or 0 to 1. In some embodiments, cₓ₋₁ to dₓ₋₁ is 8 to 10 and cₓ to dₓ is selected from 0 to 7, 0 to 6, 0 to 5, 0 to 4, 0 to 3, 0 to 2, or 0 to 1. In some embodiments, cₓ₋₁ to dₓ₋₁ is 9 to 10 and cₓ to dₓ is selected from 0 to 8, 0 to 7, 0 to 6, 0 to 5, 0 to 4, 0 to 3, 0 to 2, or 0 to 1.

In some embodiments, the x questionnaire is administered at least 24-hours after the x-1 questionnaire. In some aspects, the x questionnaire is administered at least 48-hours after the x-1 questionnaire. In some aspects, the x questionnaire is administered at least 72-hours after the x-1 questionnaire. In some aspects, the x questionnaire is administered at least 96-hours after the x-1 questionnaire. In some aspects, the x questionnaire is administered at least 120-hours after the x-1 questionnaire. In some aspects, the x questionnaire is administered at least 144-hours after the x-1 questionnaire. In some aspects, the x questionnaire is administered at least 168-hours after the x-1 questionnaire.

### 3.3. Methods of Evaluation and Treatment Comprising Scoring the Impact of Sweating on Activities During the Past 24-Hours

In some embodiments, provided herein is a method for evaluating hyperhidrosis in a subject comprising scoring the impact of the subject's sweating on their activities during the past 24-hours. In some embodiments, the scoring is on a scale of “not at all,” “a little bit,” “a moderate amount,” “a great deal,” and “an extreme amount,” or equivalents of these choices. In some embodiments, the scoring comprises administering a questionnaire to the subject. See Question 3 in Example 1 of this disclosure.

In some embodiments, provided herein is a method for treating hyperhidrosis in a subject comprising: (i) scoring the impact of the subject's sweating on their activities during the past 24-hours as e₁ in a first scoring; and (ii) administering a sufficient amount of a pharmaceutical composition comprising a therapeutically effective amount of an agent effective to treat hyperhidrosis to the subject such that after treatment with the pharmaceutical composition, the impact is e₂ in a second scoring.

In some embodiments, e₁ is selected from “an extreme amount,” “a great deal,” and “a moderate amount.” In some aspects, e₁ is selected from “an extreme amount” and “a great deal.” In some aspects, e₁ is “an extreme amount.”

In some embodiments, e₂ is selected from “a moderate amount,” “a little bit,” and “not at all.” In some aspects, e₂ is selected from “a little bit” and “not at all.” In some aspects, e₂ is “not at all.”

In some embodiments, e₁ is selected from “an extreme amount,” “a great deal,” and “a moderate amount,” and e₂ is selected from “a little bit” and “not at all.” In some embodiments, e₁ is selected from “an extreme amount” and “a great deal,” and e₂ is selected from “a moderate amount,” “a little bit,” and “not at all.” In some embodiments, e₁ is “an extreme amount,” and e₂ is selected from “a great deal,” “a moderate amount,” “a little bit,” and “not at all.”

In some embodiments, the second scoring is performed at least 24-hours after the first scoring. In some aspects, the second scoring is performed at least 48-hours after the first scoring. In some aspects, the second scoring is performed at least 72-hours after the first scoring. In some aspects, the second scoring is performed at least 96-hours after the first scoring. In some aspects, the second scoring is performed at least 120-hours after the first scoring. In some aspects, the second scoring is performed at least 144-hours after the first scoring. In some aspects, the second scoring is performed at least 168-hours after the first scoring.

In some embodiments, a third scoring is performed, wherein the third scoring comprises scoring the impact of the subject's sweating on their activities during the past 24-hours as e₃.

In some embodiments, e₃ is selected from “a moderate amount,” “a little bit,” and “not at all.” In some aspects, e₃ is selected from “a little bit” and “not at all.” In some aspects, e₃ is “not at all.”

In some embodiments, e₂ is selected from “an extreme amount,” “a great deal,” and “a moderate amount,” and e₃ is selected from “a little bit” and “not at all.” In some embodiments, e₂ is selected from “an extreme amount” and “a great deal,” and e₃ is selected from “a moderate amount,” “a little bit,” and “not at all.” In some embodiments, e₂ is “an extreme amount,” and e₃ is selected from “a great deal,” “a moderate amount,” “a little bit,” and “not at all.”

In some embodiments, the third scoring is performed at least 24-hours after the second scoring. In some aspects, the third scoring is performed at least 48-hours after the second scoring. In some aspects, the third scoring is performed at least 72-hours after the second scoring. In some aspects, the third scoring is performed at least 96-hours after the second scoring. In some aspects, the third scoring is performed at least 120-hours after the second scoring. In some aspects, the third scoring is performed at least 144-hours after the second scoring. In some aspects, the third scoring is performed at least 168-hours after the second scoring.

In some embodiments, a further scoring is performed, wherein the further scoring comprises scoring the impact of the subject's sweating on their activities during the past 24-hours as eₓ, where x is 4 or greater.

In some embodiments, eₓ is selected from “a moderate amount,” “a little bit,” and “not at all.” In some aspects, eₓ is selected from “a little bit” and “not at all.” In some aspects, eₓ is “not at all.”

In some embodiments, eₓ₋₁ is selected from “an extreme amount,” “a great deal,” and “a moderate amount,” and eₓ is selected from “a little bit” and “not at all.” In some embodiments, eₓ₋₁ is selected from “an extreme amount” and “a great deal,” and eₓ is selected from “a moderate amount,” “a little bit,” and “not at all.” In some embodiments, eₓ₋₁ is “an extreme amount,” and eₓ is selected from “a great deal,” “a moderate amount,” “a little bit,” and “not at all.”

In some embodiments, the x scoring is performed at least 24-hours after the x-1 scoring. In some aspects, the x scoring is performed at least 48-hours after the x-1 scoring. In some aspects, the x scoring is performed at least 72-hours after the x-1 scoring. In some aspects, the x scoring is performed at least 96-hours after the x-1 scoring. In some aspects, the x scoring is performed at least 120-hours after the x-1 scoring. In some aspects, the x scoring is performed at least 144-hours after the x-1 scoring. In some aspects, the x scoring is performed at least 168-hours after the x-1 scoring.

In some embodiments, one or more of the scoring steps comprises administering a questionnaire to the subject.

In some embodiments, provided herein is a method for treating hyperhidrosis in a subject comprising: (i) administering a first questionnaire to the subject before treatment with a pharmaceutical composition comprising a therapeutically effective amount of an agent effective to treat hyperhidrosis, wherein the first questionnaire comprises a question asking the subject to what extent their sweating impacted their activities during the past 24-hours, wherein the subject rates the impact on the first questionnaire as f₁; and (ii) administering a sufficient amount of the pharmaceutical composition to the subject such that on administering a second questionnaire to the subject after treatment with the pharmaceutical composition, wherein the second questionnaire comprises a question asking the subject to what extent their sweating impacted their activities during the past 24-hours, the subject rates the impact on the second questionnaire as f₂.

In some embodiments, f₁ is selected from “an extreme amount,” “a great deal,” and “a moderate amount.” In some aspects, f₁ is selected from “an extreme amount” and “a great deal.” In some aspects, f₁ is “an extreme amount.”

In some embodiments, f₂ is selected from “a moderate amount,” “a little bit,” and “not at all.” In some aspects, f₂ is selected from “a little bit” and “not at all.” In some aspects, f₂ is “not at all.”

In some embodiments, f₁ is selected from “an extreme amount,” “a great deal,” and “a moderate amount,” and f₂ is selected from “a little bit” and “not at all.” In some embodiments, f₁ is selected from “an extreme amount” and “a great deal,” and f₂ is selected from “a moderate amount,” “a little bit,” and “not at all.” In some embodiments, f₁ is “an extreme amount,” and f₂ is selected from “a great deal,” “a moderate amount,” “a little bit,” and “not at all.”

In some embodiments, the second questionnaire is administered at least 24-hours after the first questionnaire. In some aspects, the second questionnaire is administered at least 48-hours after the first questionnaire. In some aspects, the second questionnaire is administered at least 72-hours after the first questionnaire. In some aspects, the second questionnaire is administered at least 96-hours after the first questionnaire. In some aspects, the second questionnaire is administered at least 120-hours after the first questionnaire. In some aspects, the second questionnaire is administered at least 144-hours after the first questionnaire. In some aspects, the second questionnaire is administered at least 168-hours after the first questionnaire.

In some embodiments, a third questionnaire is administered to the subject, wherein the third questionnaire comprises a question asking the subject to what extent their sweating impacted their activities during the past 24-hours, the subject rates the impact on the third questionnaire as f₃.

In some embodiments, f₃ is selected from “a moderate amount,” “a little bit,” and “not at all.” In some aspects, f₃ is selected from “a little bit” and “not at all.” In some aspects, f₃ is “not at all.”

In some embodiments, f₂ is selected from “an extreme amount,” “a great deal,” and “a moderate amount,” and f₃ is selected from “a little bit” and “not at all.” In some embodiments, f₂ is selected from “an extreme amount” and “a great deal,” and f₃ is selected from “a moderate amount,” “a little bit,” and “not at all.” In some embodiments, f₂ is “an extreme amount,” and f₃ is selected from “a great deal,” “a moderate amount,” “a little bit,” and “not at all.”

In some embodiments, the third questionnaire is administered at least 24-hours after the second questionnaire. In some aspects, the third questionnaire is administered at least 48-hours after the second questionnaire. In some aspects, the third questionnaire is administered at least 72-hours after the second questionnaire. In some aspects, the third questionnaire is administered at least 96-hours after the second questionnaire. In some aspects, the third questionnaire is administered at least 120-hours after the second questionnaire. In some aspects, the third questionnaire is administered at least 144-hours after the second questionnaire. In some aspects, the third questionnaire is administered at least 168-hours after the second questionnaire.

In some embodiments, a further questionnaire is administered to the subject, wherein the further questionnaire comprises a question asking the subject to what extent their sweating impacted their activities during the past 24-hours, the subject rates the impact on the further questionnaire as fₓ, where x is 4 or greater.

In some embodiments, fₓ is selected from “a moderate amount,” “a little bit,” and “not at all.” In some aspects, fₓ is selected from “a little bit” and “not at all.” In some aspects, fₓ is “not at all.”

In some embodiments, fₓ₋₁ is selected from “an extreme amount,” “a great deal,” and “a moderate amount,” and fₓ is selected from “a little bit” and “not at all.” In some embodiments, fₓ₋₁ is selected from “an extreme amount” and “a great deal,” and fₓ is selected from “a moderate amount,” “a little bit,” and “not at all.” In some embodiments, fₓ₋₁ is “an extreme amount,” and fₓ is selected from “a great deal,” “a moderate amount,” “a little bit,” and “not at all.”

In some embodiments, the x questionnaire is administered at least 24-hours after the x-1 questionnaire. In some aspects, the x questionnaire is administered at least 48-hours after the x-1 questionnaire. In some aspects, the x questionnaire is administered at least 72-hours after the x-1 questionnaire. In some aspects, the x questionnaire is administered at least 96-hours after the x-1 questionnaire. In some aspects, the x questionnaire is administered at least 120-hours after the x-1 questionnaire. In some aspects, the x questionnaire is administered at least 144-hours after the x-1 questionnaire. In some aspects, the x questionnaire is administered at least 168-hours after the x-1 questionnaire.

### 3.4. Methods of Evaluation and Treatment Comprising Scoring how Bothered a Subject was by Sweating During the Past 24-Hours

In some embodiments, provided herein is a method for evaluating hyperhidrosis in a subject comprising scoring how bothered the subject was by their sweating during the past 24-hours. In some embodiments, the scoring is on a scale of “not at all bothered,” “a little bothered,” “moderately bothered,” “very bothered,” and “extremely bothered,” or equivalents of these choices. In some embodiments, the scoring comprises administering a questionnaire to the subject. See Question 4 in Example 1 of this disclosure.

In some embodiments, provided herein is a method for treating hyperhidrosis in a subject comprising: (i) scoring how bothered the subject was by their sweating during the past 24-hours as g₁ in a first scoring; and (ii) administering a sufficient amount of a pharmaceutical composition comprising a therapeutically effective amount of an agent effective to treat hyperhidrosis to the subject, such that after treatment with the pharmaceutical composition, the subject is bothered by an amount g₂ in a second scoring.

In some embodiments, g₁ is selected from “extremely bothered,” “very bothered,” and “moderately bothered.” In some aspects, g₁ is selected from “extremely bothered” and “very bothered.” In some aspects, g₁ is “extremely bothered.”

In some embodiments, g₂ is selected from “moderately bothered,” “a little bothered,” and “not at all bothered.” In some aspects, g₂ is selected from “a little bothered” and “not at all bothered.” In some aspects, g₂ is “not at all bothered.”

In some embodiments, g₁ is selected from “extremely bothered,” “very bothered,” and “moderately bothered,” and g₂ is selected from “a little bothered” and “not at all bothered.” In some embodiments, g₁ is selected from “extremely bothered” and “very bothered,” and g₂ is selected from “moderately bothered,” “a little bothered,” and “not at all bothered.” In some embodiments, g₁ is “extremely bothered,” and g₂ is selected from “very bothered,” “moderately bothered,” “a little bothered,” and “not at all bothered.”

In some embodiments, the second scoring is performed at least 24-hours after the first scoring. In some aspects, the second scoring is performed at least 48-hours after the first scoring. In some aspects, the second scoring is performed at least 72-hours after the first scoring. In some aspects, the second scoring is performed at least 96-hours after the first scoring. In some aspects, the second scoring is performed at least 120-hours after the first scoring. In some aspects, the second scoring is performed at least 144-hours after the first scoring. In some aspects, the second scoring is performed at least 168-hours after the first scoring.

In some embodiments, a third scoring is performed, wherein the third scoring comprises scoring how bothered the subject was by their sweating during the past 24-hours as g₃.

In some embodiments, g₃ is selected from “moderately bothered,” “a little bothered,” and “not at all bothered.” In some aspects, g₃ is selected from “a little bothered” and “not at all bothered.” In some aspects, g₃ is “not at all bothered.”

In some embodiments, g₂ is selected from “extremely bothered,” “very bothered,” and “moderately bothered,” and g₃ is selected from “a little bothered” and “not at all bothered.” In some embodiments, g₂ is selected from “extremely bothered” and “very bothered,” and g₃ is selected from “moderately bothered,” “a little bothered,” and “not at all bothered.” In some embodiments, g₂ is “extremely bothered,” and g₃ is selected from “very bothered,” “moderately bothered,” “a little bothered,” and “not at all bothered.”

In some embodiments, the third scoring is performed at least 24-hours after the second scoring. In some aspects, the third scoring is performed at least 48-hours after the second scoring. In some aspects, the third scoring is performed at least 72-hours after the second scoring. In some aspects, the third scoring is performed at least 96-hours after the second scoring. In some aspects, the third scoring is performed at least 120-hours after the second scoring. In some aspects, the third scoring is performed at least 144-hours after the second scoring. In some aspects, the third scoring is performed at least 168-hours after the second scoring.

In some embodiments, a further scoring is performed, wherein the further scoring comprises scoring how bothered the subject was by their sweating during the past 24-hours as gₓ, where x is 4 or greater.

In some embodiments, gₓ is selected from “moderately bothered,” “a little bothered,” and “not at all bothered.” In some aspects, gₓ is selected from “a little bothered” and “not at all bothered.” In some aspects, gₓ is “not at all bothered.”

In some embodiments, gₓ₋₁ is selected from “extremely bothered,” “very bothered,” and “moderately bothered,” and gₓ is selected from “a little bothered” and “not at all bothered.” In some embodiments, gₓ₋₁ is selected from “extremely bothered” and “very bothered,” and gₓ is selected from “moderately bothered,” “a little bothered,” and “not at all bothered.” In some embodiments, gₓ₋₁ is “extremely bothered,” and gₓ is selected from “very bothered,” “moderately bothered,” “a little bothered,” and “not at all bothered.”

In some embodiments, the x scoring is performed at least 24-hours after the x-1 scoring. In some aspects, the x scoring is performed at least 48-hours after the x-1 scoring. In some aspects, the x scoring is performed at least 72-hours after the x-1 scoring. In some aspects, the x scoring is performed at least 96-hours after the x-1 scoring. In some aspects, the x scoring is performed at least 120-hours after the x-1 scoring. In some aspects, the x scoring is performed at least 144-hours after the x-1 scoring. In some aspects, the x scoring is performed at least 168-hours after the x-1 scoring.

In some embodiments, one or more of the scoring steps comprises administering a questionnaire to the subject.

In some embodiments, provided herein is a method for treating hyperhidrosis in a subject comprising: (i) administering a first questionnaire to the subject before treatment with a pharmaceutical composition comprising a therapeutically effective amount of an agent effective to treat hyperhidrosis, wherein the first questionnaire comprises a question asking the subject how bothered they were by their sweating, during the past 24-hours, wherein the subject reports that they were bothered by an amount h₁ in the first questionnaire; and (ii) administering a sufficient amount of the pharmaceutical composition to the subject such that on administering a second questionnaire to the subject after treatment with the pharmaceutical composition, wherein the second questionnaire comprises a question asking the subject how bothered they were by their sweating, during the past 24-hours, the subject reports that they were bothered by an amount h₂ in the second questionnaire.

In some embodiments, h₁ is selected from “extremely bothered,” “very bothered,” and “moderately bothered.” In some aspects, h₁ is selected from “extremely bothered” and “very bothered.” In some aspects, h₁ is “extremely bothered.”

In some embodiments, h₂ is selected from “moderately bothered,” “a little bothered,” and “not at all bothered.” In some aspects, h₂ is selected from “a little bothered” and “not at all bothered.” In some aspects, h₂ is “not at all bothered.”

In some embodiments, h₁ is selected from “extremely bothered,” “very bothered,” and “moderately bothered,” and h₂ is selected from “a little bothered” and “not at all bothered.” In some embodiments, h₁ is selected from “extremely bothered” and “very bothered,” and h₂ is selected from “moderately bothered,” “a little bothered,” and “not at all bothered.” In some embodiments, h₁ is “extremely bothered,” and h₂ is selected from “very bothered,” “moderately bothered,” “a little bothered,” and “not at all bothered.”

In some embodiments, the second questionnaire is administered at least 24-hours after the first questionnaire. In some aspects, the second questionnaire is administered at least 48-hours after the first questionnaire. In some aspects, the second questionnaire is administered at least 72-hours after the first questionnaire. In some aspects, the second questionnaire is administered at least 96-hours after the first questionnaire. In some aspects, the second questionnaire is administered at least 120-hours after the first questionnaire. In some aspects, the second questionnaire is administered at least 144-hours after the first questionnaire. In some aspects, the second questionnaire is administered at least 168-hours after the first questionnaire.

In some embodiments, a third questionnaire is administered to the subject, wherein the third questionnaire comprises a question asking the subject how bothered they were by their sweating, during the past 24-hours, the subject reports that they were bothered by an amount h₃ in the third questionnaire.

In some embodiments, h₃ is selected from “moderately bothered,” “a little bothered,” and “not at all bothered.” In some aspects, h₃ is selected from “a little bothered” and “not at all bothered.” In some aspects, h₃ is “not at all bothered.”

In some embodiments, h₂ is selected from “extremely bothered,” “very bothered,” and “moderately bothered,” and h₃ is selected from “a little bothered” and “not at all bothered.” In some embodiments, h₂ is selected from “extremely bothered” and “very bothered,” and h₃ is selected from “moderately bothered,” “a little bothered,” and “not at all bothered.” In some embodiments, h₂ is “extremely bothered,” and h₃ is selected from “very bothered,” “moderately bothered,” “a little bothered,” and “not at all bothered.”

In some embodiments, the third questionnaire is administered at least 24-hours after the second questionnaire. In some aspects, the third questionnaire is administered at least 48-hours after the second questionnaire. In some aspects, the third questionnaire is administered at least 72-hours after the second questionnaire. In some aspects, the third questionnaire is administered at least 96-hours after the second questionnaire. In some aspects, the third questionnaire is administered at least 120-hours after the second questionnaire. In some aspects, the third questionnaire is administered at least 144-hours after the second questionnaire. In some aspects, the third questionnaire is administered at least 168-hours after the second questionnaire.

In some embodiments, a further questionnaire is administered to the subject, wherein the further questionnaire comprises a question asking the subject how bothered they were by their sweating, during the past 24-hours, the subject reports that they were bothered by an amount hₓ in the further questionnaire, where x is 4 or greater.

In some embodiments, hₓ is selected from “moderately bothered,” “a little bothered,” and “not at all bothered.” In some aspects, hₓ is selected from “a little bothered” and “not at all bothered.” In some aspects, hₓ is “not at all bothered.”

In some embodiments, hₓ₋₁ is selected from “extremely bothered,” “very bothered,” and “moderately bothered,” and hₓ is selected from “a little bothered” and “not at all bothered.” In some embodiments, hₓ₋₁ is selected from “extremely bothered” and “very bothered,” and hₓ is selected from “moderately bothered,” “a little bothered,” and “not at all bothered.” In some embodiments, hₓ₋₁ is “extremely bothered,” and hₓ is selected from “very bothered,” “moderately bothered,” “a little bothered,” and “not at all bothered.”

In some embodiments, the x questionnaire is administered at least 24-hours after the x-1 questionnaire. In some aspects, the x questionnaire is administered at least 48-hours after the x-1 questionnaire. In some aspects, the x questionnaire is administered at least 72-hours after the x-1 questionnaire. In some aspects, the x questionnaire is administered at least 96-hours after the x-1 questionnaire. In some aspects, the x questionnaire is administered at least 120-hours after the x-1 questionnaire. In some aspects, the x questionnaire is administered at least 144-hours after the x-1 questionnaire. In some aspects, the x questionnaire is administered at least 168-hours after the x-1 questionnaire.

### 3.5. Scoring Sweating Symptoms Experienced by the Subject Over a Defined Period

In some embodiments, provided herein is a method for evaluating hyperhidrosis in a subject comprising scoring whether the subject exhibited 3 or more symptoms of hyperhidrosis during the past 7 days. In some embodiments, the 3 or more symptoms comprise the following symptoms: (a) having to change an article of clothing (e.g., a shirt) during the day because of sweating; (b) having to take more than one shower or bath during the day because of sweating; (c) feeling less confident in themselves because of sweating; (d) feeling embarrassed by their sweating; (e) avoiding interactions with other people because of their sweating; and (f) not doing an activity that they wanted or needed to do because of their sweating; or equivalents of these symptoms. In some embodiments, the scoring comprises administering a questionnaire to the subject. See Weekly Impact Items in Example 2 of this disclosure.

In some embodiments, provided herein is a method for treating hyperhidrosis in a subject comprising: (i) scoring whether the subject exhibited i symptoms of hyperhidrosis during the past j days at a value of at least k₁% of the symptoms in a first scoring; and (ii) administering a sufficient amount of a pharmaceutical composition comprising a therapeutically effective amount of an agent effective to treat hyperhidrosis to the subject such that after treatment with the pharmaceutical composition, the subject exhibits k₂% or fewer of the symptoms in a second scoring.

In some embodiments, the number of questions, i, is 6. In some embodiments, the number of questions, i, is 3, 4, 5, 7, 8, 9, 10, or more.

In some embodiments, the number of days, j, is 7. In some embodiments, the number of days, j, is selected from 1, 2, 3, 4, 5, 6, 8, 9, 10, or more.

In some embodiments, the percent of the symptoms exhibited by the subject in the first scoring, k₁, is selected from at least 50%, at least 60%, at least 70%, at least 80%, or at least 90%.

In some embodiments, the percent of the symptoms exhibited by the subject on the second scoring, k₂, is selected from 50% or fewer, 40% or fewer, 30% or fewer, 20% or fewer, or 10% or fewer.

In some embodiments, the number of questions, i, is 6 and the subject exhibits at least 4 of the symptoms in the first scoring. In some aspects, the subject exhibits at least 5 of the symptoms in the first scoring. In some aspects, the subject exhibits all 6 of the symptoms in the first scoring.

In some embodiments, the number of questions, i, is 6 and the subject exhibits 4 or fewer of the symptoms in the second scoring. In some aspects, the subject exhibits 3 or fewer of the symptoms in the second scoring. In some aspects, the subject exhibits 2 or fewer of the symptoms in the second scoring. In some aspects, the subject exhibits 1 or fewer of the symptoms in the second scoring. In some aspects, the subject exhibits none of the symptoms in the second scoring.

In some embodiments, the number of questions, i, is 6 and the subject exhibits at least 4, 5, or 6 of the symptoms in the first scoring, and 3, 2, 1, or 0 of the symptoms in the second scoring. In some embodiments, the number of questions, i, is 6 and the subject exhibits at least 5, or 6 of the symptoms in the first scoring, and 4, 3, 2, 1, or 0 of the symptoms in the second scoring. In some embodiments, the number of questions, i, is 6 and the subject exhibits all 6 of the symptoms in the first scoring, and 5, 4, 3, 2, 1, or 0 of the symptoms in the second scoring.

In some embodiments, the second scoring is performed at least 7 days after the first scoring. In some aspects, the second scoring is performed at least 14 days after the first scoring. In some aspects, the second scoring is performed at least 21 days after the first scoring. In some aspects, the second scoring is performed at least 28 days after the first scoring. In some aspects, the second scoring is performed at least one month after the first scoring.

In some embodiments, a third scoring is performed, wherein the subject exhibits k₃% or fewer of the symptoms in the third scoring.

In some embodiments, the percent of the symptoms exhibited by the subject in the third scoring, k₃, is selected from 50% or fewer, 40% or fewer, 30% or fewer, 20% or fewer, or 10% or fewer.

In some embodiments, the number of questions, i, is 6 and the subject exhibits 4 or fewer of the symptoms in the third scoring. In some aspects, the subject exhibits 3 or fewer of the symptoms in the third scoring. In some aspects, the subject exhibits 2 or fewer of the symptoms in the third scoring. In some aspects, the subject exhibits 1 or fewer of the symptoms in the third scoring. In some aspects, the subject exhibits none of the symptoms in the third scoring.

In some embodiments, the number of questions, i, is 6 and the subject exhibits at least 4, 5, or 6 of the symptoms in the second scoring, and 3, 2, 1, or 0 of the symptoms in the third scoring. In some embodiments, the number of questions, i, is 6 and the subject exhibits at least 5, or 6 of the symptoms in the second scoring, and 4, 3, 2, 1, or 0 of the symptoms in the third scoring. In some embodiments, the number of questions, i, is 6 and the subject exhibits all 6 of the symptoms in the second scoring, and 5, 4, 3, 2, 1, or 0 of the symptoms in the third scoring.

In some embodiments, the third scoring is performed at least 7 days after the second scoring. In some aspects, the third scoring is performed at least 14 days after the second scoring. In some aspects, the third scoring is performed at least 21 days after the second scoring. In some aspects, the third scoring is performed at least 28 days after the second scoring. In some aspects, the third scoring is performed at least one month after the second scoring.

In some embodiments, a further questionnaire is performed, wherein the subject exhibits kₓ% or fewer of the symptoms in the further scoring, where x is 4 or greater.

In some embodiments, the percent of the symptoms exhibited by the subject in the further scoring, kₓ, is selected from 50% or fewer, 40% or fewer, 30% or fewer, 20% or fewer, or 10% or fewer.

In some embodiments, the number of questions, i, is 6 and the subject exhibits 4 or fewer of the symptoms in the further scoring. In some aspects, the subject exhibits 3 or fewer of the symptoms in the further scoring. In some aspects, the subject exhibits 2 or fewer of the symptoms in the further scoring. In some aspects, the subject exhibits 1 or fewer of the symptoms in the further scoring. In some aspects, the subject exhibits none of the symptoms in the further scoring.

In some embodiments, the number of questions, i, is 6 and the subject exhibits at least 4, 5, or 6 of the symptoms in the x-1 scoring, and 3, 2, 1, or 0 of the symptoms in the x scoring. In some embodiments, the number of questions, i, is 6 and the subject exhibits at least 5, or 6 of the symptoms in the x-1 questionnaire, and 4, 3, 2, 1, or 0 of the symptoms in the x scoring. In some embodiments, the number of questions, i, is 6 and the subject exhibits all 6 of the symptoms in the x-1 scoring, and 5, 4, 3, 2, 1, or 0 of the symptoms in the x scoring.

In some embodiments, the x scoring is performed at least 7 days after the x-1 scoring. In some aspects, the x scoring is performed at least 14 days after the x-1 scoring. In some aspects, the x scoring is performed at least 21 days after the x-1 scoring. In some aspects, the x scoring is performed at least 28 days after the x-1 scoring. In some aspects, the x scoring is performed at least one month after the x-1 scoring.

In some embodiments, one or more of the scoring steps comprises administering a questionnaire to the subject.

In some embodiments, provided herein is a method for treating hyperhidrosis in a subject comprising: (i) administering a first questionnaire to the subject before treatment with a pharmaceutical composition comprising a therapeutically effective amount of an agent effective to treat hyperhidrosis, wherein the first questionnaire comprises a battery of 1 questions, each of the questions asking whether the subject has or has not experienced a symptom of hyperhidrosis within the past m days, and wherein the subject reports experiencing at least n₁% of the symptoms on the first questionnaire; and (ii) administering a sufficient amount of the pharmaceutical composition to the subject such that on administering a second questionnaire to the subject after treatment with the pharmaceutical composition, wherein the second questionnaire comprises the battery of questions, and the subject reports experiencing n₂% or fewer of the symptoms on the second questionnaire.

In some embodiments, the number of questions, l, is 6. In some embodiments, the number of questions, l, is 3, 4, 5, 7, 8, 9, 10, or more.

In some embodiments, the number of days, m, is 7. In some embodiments, the number of days, m, is selected from 1, 2, 3, 4, 5, 6, 8, 9, 10, or more.

In some embodiments, the percent of the symptoms exhibited by the subject on the first questionnaire, n₁ is selected from at least 50%, at least 60%, at least 70%, at least 80%, or at least 90%.

In some embodiments, the percent of the symptoms exhibited by the subject on the second questionnaire, n₂, is selected from 50% or fewer, 40% or fewer, 30% or fewer, 20% or fewer, or 10% or fewer.

In some embodiments, the number of questions, l, is 6 and the subject reports experiencing at least 4 of the symptoms inquired about on the first questionnaire. In some aspects, the subject reports experiencing at least 5 of the symptoms inquired about on the first questionnaire. In some aspects, the subject reports experiencing all 6 of the symptoms inquired about on the first questionnaire.

In some embodiments, the number of questions, l, is 6 and the subject reports experiencing 4 or fewer of the symptoms inquired about on the second questionnaire. In some aspects, the subject reports experiencing 3 or fewer of the symptoms inquired about on the second questionnaire. In some aspects, the subject reports experiencing 2 or fewer of the symptoms inquired about on the second questionnaire. In some aspects, the subject reports experiencing 1 or fewer of the symptoms inquired about on the second questionnaire. In some aspects, the subject reports experiencing none of the symptoms inquired about on the second questionnaire.

In some embodiments, the number of questions, l, is 6 and the subject reports experiencing at least 4, 5, or 6 of the symptoms inquired about on the first questionnaire, and 3, 2, 1, or 0 of the symptoms inquired about on the second questionnaire. In some embodiments, the number of questions, l, is 6 and the subject reports experiencing at least 5, or 6 of the symptoms inquired about on the first questionnaire, and 4, 3, 2, 1, or 0 of the symptoms inquired about on the second questionnaire. In some embodiments, the number of questions, l, is 6 and the subject reports experiencing all 6 of the symptoms inquired about on the first questionnaire, and 5, 4, 3, 2, 1, or 0 of the symptoms inquired about on the second questionnaire.

In some embodiments, the second questionnaire is administered at least 7 days after the first questionnaire. In some aspects, the second questionnaire is administered at least 14 days after the first questionnaire. In some aspects, the second questionnaire is administered at least 21 days after the first questionnaire. In some aspects, the second questionnaire is administered at least 28 days after the first questionnaire. In some aspects, the second questionnaire is administered at least one month after the first questionnaire.

In some embodiments, a third questionnaire is administered to the subject, wherein the third questionnaire comprises the battery of questions, and the subject reports experiencing n₃% or fewer of the symptoms on the third questionnaire.

In some embodiments, the percent of the symptoms exhibited by the subject on the third questionnaire, n₃, is selected from 50% or fewer, 40% or fewer, 30% or fewer, 20% or fewer, or 10% or fewer.

In some embodiments, the number of questions, l, is 6 and the subject reports experiencing 4 or fewer of the symptoms inquired about on the third questionnaire. In some aspects, the subject reports experiencing 3 or fewer of the symptoms inquired about on the third questionnaire. In some aspects, the subject reports experiencing 2 or fewer of the symptoms inquired about on the third questionnaire. In some aspects, the subject reports experiencing 1 or fewer of the symptoms inquired about on the third questionnaire. In some aspects, the subject reports experiencing none of the symptoms inquired about on the third questionnaire.

In some embodiments, the number of questions, l, is 6 and the subject reports experiencing at least 4, 5, or 6 of the symptoms inquired about on the second questionnaire, and 3, 2, 1, or 0 of the symptoms inquired about on the third questionnaire. In some embodiments, the number of questions, l, is 6 and the subject reports experiencing at least 5, or 6 of the symptoms inquired about on the second questionnaire, and 4, 3, 2, 1, or 0 of the symptoms inquired about on the third questionnaire. In some embodiments, the number of questions, l, is 6 and the subject reports experiencing all 6 of the symptoms inquired about on the second questionnaire, and 5, 4, 3, 2, 1, or 0 of the symptoms inquired about on the third questionnaire.

In some embodiments, the third questionnaire is administered at least 7 days after the second questionnaire. In some aspects, the third questionnaire is administered at least 14 days after the second questionnaire. In some aspects, the third questionnaire is administered at least 21 days after the second questionnaire. In some aspects, the third questionnaire is administered at least 28 days after the second questionnaire. In some aspects, the third questionnaire is administered at least one month after the second questionnaire.

In some embodiments, a further questionnaire is administered to the subject, wherein the further questionnaire comprises the battery of questions, and the subject reports experiencing nₓ% or fewer of the symptoms on the further questionnaire, where x is 4 or greater.

In some embodiments, the percent of the symptoms exhibited by the subject on the third questionnaire, nₓ, is selected from 50% or fewer, 40% or fewer, 30% or fewer, 20% or fewer, or 10% or fewer.

In some embodiments, the number of questions, l, is 6 and the subject reports experiencing 4 or fewer of the symptoms inquired about on the further questionnaire. In some aspects, the subject reports experiencing 3 or fewer of the symptoms inquired about on the further questionnaire. In some aspects, the subject reports experiencing 2 or fewer of the symptoms inquired about on the further questionnaire. In some aspects, the subject reports experiencing 1 or fewer of the symptoms inquired about on the further questionnaire. In some aspects, the subject reports experiencing none of the symptoms inquired about on the further questionnaire.

In some embodiments, the number of questions, l, is 6 and the subject reports experiencing at least 4, 5, or 6 of the symptoms inquired about on the x-1 questionnaire, and 3, 2, 1, or 0 of the symptoms inquired about on the x questionnaire. In some embodiments, the number of questions, l, is 6 and the subject reports experiencing at least 5, or 6 of the symptoms inquired about on the x-1 questionnaire, and 4, 3, 2, 1, or 0 of the symptoms inquired about on the x questionnaire. In some embodiments, the number of questions, l, is 6 and the subject reports experiencing all 6 of the symptoms inquired about on the x-1 questionnaire, and 5, 4, 3, 2, 1, or 0 of the symptoms inquired about on the x questionnaire.

In some embodiments, the x questionnaire is administered at least 7 days after the x-1 questionnaire. In some aspects, the x questionnaire is administered at least 14 days after the x-1 questionnaire. In some aspects, the x questionnaire is administered at least 21 days after the x-1 questionnaire. In some aspects, the x questionnaire is administered at least 28 days after the x-1 questionnaire. In some aspects, the x questionnaire is administered at least one month after the x-1 questionnaire.

### 3.6. Scoring a Subject's Global Impression of Change after Treatment

In some embodiments, provided herein is a method for evaluating hyperhidrosis in a subject comprising scoring the subject's overall underarm sweating at present, as compared to before treatment. In some embodiments, the scoring is on a scale of “much better,” “moderately better,” “a little better,” “no difference,” “a little worse,” “moderately worse,” “much worse,” or equivalents of these choices. In some embodiments, the scoring comprises administering a questionnaire to the subject. See Patient Global Impression of Change Item in Example 3 of this disclosure.

In some embodiments, provided herein is a method for treating hyperhidrosis in a subject comprising: (i) administering a sufficient amount of a pharmaceutical composition comprising a therapeutically effective amount of an agent effective to treat hyperhidrosis to the subject; and (ii) scoring the subject's overall sweating at present, as compared to before treatment, as o₁ in a first scoring.

In some embodiments, o₁ is selected from “a little better,” “moderately better,” or much better.” In some aspects, o₁ is selected from “moderately better” or “much better.” In some aspects, o₁ is “much better.”

In some embodiments, a second scoring is performed. In some embodiments, at least 7 days pass between the first scoring and the second scoring. In some embodiments, at least 14 days pass between the first scoring and the second scoring. In some embodiments, at least 21 days pass between the first scoring and the second scoring. In some embodiments, at least 28 days pass between the first scoring and the second scoring. In some embodiments, at least one month passes between the first scoring and the second scoring. In some embodiments, at least three months pass between the first scoring and the second scoring. In some embodiments, at least six months pass between the first scoring and the second scoring. In some embodiments, at least one year passes between the first scoring and the second scoring.

In some embodiments, the subject's overall sweating at present, as compared to before treatment, is oₓ in a further scoring, where x is 2 or greater.

In some embodiments, oₓ is selected from “a little better,” “moderately better,” or much better.” In some aspects, oₓ is selected from “moderately better” or “much better.” In some aspects, oₓ is “much better.”

In some embodiments, provided herein is a method for treating hyperhidrosis in a subject comprising: (i) administering a pharmaceutical composition comprising a therapeutically effective amount of an agent effective to treat hyperhidrosis to the subject; and administering a questionnaire to the subject after treatment with the pharmaceutical composition, wherein the questionnaire comprises a question asking the subject to rate their overall underarm sweating at present, as compared to before treatment, and wherein the subject rates their overall underarm sweating at present, as compared to before treatment, as p₁.

In some embodiments, p₁ is selected from “a little better,” “moderately better,” or much better.” In some aspects, p₁ is selected from “moderately better” or “much better.” In some aspects, p₁ is “much better.”

In some embodiments, a second questionnaire is administered to the subject. In some embodiments, at least 7 days pass between the first administration of the questionnaire and the second administration of the questionnaire. In some embodiments, at least 14 days pass between the first administration of the questionnaire and the second administration of the questionnaire. In some embodiments, at least 21 days pass between the first administration of the questionnaire and the second administration of the questionnaire. In some embodiments, at least 28 days pass between the first administration of the questionnaire and the second administration of the questionnaire. In some embodiments, at least one month passes between the first administration of the questionnaire and the second administration of the questionnaire. In some embodiments, at least three months pass between the first administration of the questionnaire and the second administration of the questionnaire. In some embodiments, at least six months pass between the first administration of the questionnaire and the second administration of the questionnaire. In some embodiments, at least one year passes between the first administration of the questionnaire and the second administration of the questionnaire.

In some embodiments, the subject rates their overall sweating at present, as compared to before treatment, as pₓ, where x is 2 or greater. In some embodiments, pₓ is selected from “a little better,” “moderately better,” or much better.” In some aspects, pₓ is selected from “moderately better” or “much better.” In some aspects, pₓ is “much better.”

### 4. Agents and Procedures for Treating Hyperhidrosis

Any suitable agent for the treatment of hyperhidrosis may be used with in the methods provided herein. Useful agents include, but are not limited to, agents for the treatment of primary hyperhidrosis.

In some embodiments, the agent is selected from an anticholinergic agent, a metal salt (e.g., aluminum chloride), or a toxin (e.g., botulinum toxin type A (Botox®)). In some embodiments, the toxin is injected. In some embodiments, the toxin is applied topically.

In some embodiments, the anticholinergic agent is selected from a glycopyrronium compound, propantheline, oxybutynin, methantheline, benztropine, and BBI-4000 (Brickell Biotech, Inc.). In some embodiments, the agent is a glycopyrronium compound. In some embodiments, the glycopyrronium compound is glycopyrronium tosylate. In some embodiments, the glycopyrronium compound is glycopyrronium bromide.

In some embodiments, the glycopyrronium base is threo-glycopyrronium tosylate. In some embodiments, the glycopyrronium base is threo-glycopyrronium bromide.

In some embodiments, the glycopyrronium compound comprises threo-glycopyrronium tosylate. In some embodiments, the glycopyrronium compound comprises threo-glycopyrronium bromide.

In some embodiments, the glycopyrronium compound comprises threo-glycopyrronium and erythro-glycopyrronium, wherein the threo-glycopyrronium is at least 95% of the total glycopyrronium content of the composition and the erythro-glycopyrronium is less than 5% of the total glycopyrronium content of the composition.

In some embodiments, the methods provided herein may further comprise administration of one or more additional agents to treat hyperhidrosis. Illustrative additional agents include any of those described in this disclosure or known in the art for the treatment of hyperhidrosis. The additional agent(s) may be administered in the same pharmaceutical composition as the agent recited in the methods provided herein, or in a different pharmaceutical composition, according to the judgment of those of skill in the art.

In some embodiments, the methods provided herein may further comprise performance of one or more procedures for treating hyperhidrosis. Illustrative procedures include those described in this disclosure and known in the art, including surgical procedures and iontophoresis.

In some embodiments, the methods provided herein comprises administering a glycopyrronium compound in combination with another agent or procedure. In some aspects, the other agent or procedure is selected from an anticholinergic agent, a metal salt, a toxin, sweat gland suction, and iontophoresis.

In some embodiments, topical administration of an agent is combined with systemic administration of the same agent, or a different agent. In some aspects, topical administration of a glycopyrronium compound is combined with systemic administration of the glycopyrronium compound.

### 5. Pharmaceutical Compositions and Methods of Administration

The glycopyrronium compounds can be formulated into pharmaceutical compositions using methods available in the art and those disclosed herein. Any of the glycopyrronium compounds described herein or known in the art can be provided in the appropriate pharmaceutical composition and be administered by a suitable route of administration.

The methods provided herein encompass administering pharmaceutical compositions containing at least one glycopyrronium compound described herein or known in the art, either used alone or in the form of a combination with one or more compatible and pharmaceutically acceptable carriers, such as diluents or adjuvants, or with another therapeutic agent.

In certain embodiments, a second agent can be formulated or packaged with a glycopyrronium compound. In some embodiments, the second agent will be formulated with the glycopyrronium compound when, according to the judgment of those of skill in the art, such co-formulation does not substantially interfere with the activity of the glycopyrronium compound and the second agent or the method of administration. In certain embodiments, the glycopyrronium compound provided herein and the second agent are formulated separately. They can be packaged together, or packaged separately, for the convenience of the practitioner of skill in the art.

In clinical practice glycopyrronium compounds may be administered by any conventional route. In certain embodiments, the glycopyrronium compound is administered topically. In certain embodiments, the glycopyrronium compound is administered topically with a wipe.

In certain embodiments, a composition provided herein is a pharmaceutical composition. Pharmaceutical compositions provided herein comprise a prophylactically or therapeutically effective amount of one or more prophylactic or therapeutic agents (e.g., a glycopyrronium compound, or other prophylactic or therapeutic agent), and typically one or more pharmaceutically acceptable carriers or excipients. In a specific embodiment and in this context, the term “pharmaceutically acceptable” means approved by a regulatory agency of a government, or listed in the U.S. Pharmacopeia or other generally recognized pharmacopeia for use in animals, and more particularly in humans.

The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Typical dosage forms comprising a glycopyrronium compound, or a pharmaceutically acceptable solvate or hydrate thereof, lie within the range of from about 50 mg to about 150 mg of active compound per day, given as a single once-a-day dose in the morning or as divided doses throughout the day. Particular dosage forms can have about 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 110, 115, 120, 125, 130, 135, 140, 145, or 150 mg of the active compound.

### 6. Topical Dosage Forms

Also provided are topical dosage forms. Topical dosage forms include, but are not limited to, sprays, aerosols, creams, lotions, ointments, gels, solutions, emulsions, suspensions, or other forms known to one of skill in the art. See, e.g., Remington's Pharmaceutical Sciences, 16^{th}, 18^{th}, 20^{th}, and 22^{nd} eds., Mack Publishing, Easton Pa. (1980, 1990, 2000 & 2012); and Introduction to Pharmaceutical Dosage Forms, 4th ed., Lea & Febiger, Philadelphia (1985).

Suitable excipients (e.g., carriers and diluents) and other materials that can be used to provide topical dosage forms encompassed herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3 diol, isopropyl myristate, isopropyl palmitate, mineral oil, and mixtures thereof to form lotions, tinctures, creams, emulsions, gels or ointments, which are nontoxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th}, 18^{th}, 20^{th}, and 22^{nd} eds., Mack Publishing, Easton Pa. (1980, 1990, 2000 & 2012).

In an embodiment, provided herein is a topical dosage form comprising about 50-150 mg of glycopyrronium compound in an alcohol:water solution and with a pH buffering agent. In an embodiment, the glycopyrronium compound is present at a concentration of about 0.25-6% (w/w). In some embodiments, the glycopyrronium compound is present at a concentration of about 1, 2, 3, or 4% (w/w). In some embodiments, the glycopyrronium compound is present at a concentration of about 3% (w/w). In an embodiment, the topical dosage form comprises about 70-105 mg of the glycopyrronium compound. In an embodiment, the topical dosage form comprises about 70 mg of the glycopyrronium compound. In an embodiment, the topical dosage form comprises about 105 mg of the glycopyrronium compound. In an embodiment, the alcohol:water ratio of the topical dosage form is selected over the range of 50:50 to 70:30, preferably over the range of 53:47 to 58:42. In an embodiment, the buffering agent is about 0.2 to 0.5% of the topical dosage form. In an embodiment, the buffering agent of the topical dosage form is citric acid/sodium citrate. In an embodiment, the pH of the topical dosage form is selected over the range of 4.0 to 5.0. In an embodiment, the pH of the topical dosage form is about 4.5.

In an embodiment, the topical dosage form is provided in a wipe for topical administration. In an embodiment, the topical dosage form is provided in a wipe soaked with the topical dosage form. In an embodiment, the topical dosage form is provided in a wipe soaked with the topical dosage form provided in a package comprising several wipes per package.

### 7. Dosage and Unit Dosage Forms

In human therapeutics, the doctor will determine the posology which he considers most appropriate according to a preventive or curative treatment and according to the age, weight, stage of the disorder and other factors specific to the subject to be treated. Alternatively, the posology will be provided on or in packaging provided in a kit comprising a dosage form. In certain embodiments, doses are from about 50 mg to about 150 mg active compound per day for an adult. Particular dosage forms can have about 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 110, 115, 120, 125, 130, 135, 140, 145, or 150 mg of a glycopyrronium compound.

In further aspects, provided are methods of treating or preventing a disorder of a subject by administering, to a subject in need thereof, an effective amount of a glycopyrronium compound, or a pharmaceutically acceptable solvate or hydrate thereof. The amount of the glycopyrronium compound which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, administration of the same composition may be repeated and the administrations may be separated by at least 1 day, 2 days, 3 days, 5 days, 10 days, 15 days, 30 days, 45 days, 2 months, 75 days, 3 months, 6 months, or more. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, 6 months, or more.

In some embodiments, an effective amount of a glycopyrronium compound is administered once daily for four weeks.

### 8. Kits

Also provided herein are kits for use in the methods of treating hyperhidrosis provided herein. In some embodiments, the kit comprises a pharmaceutical composition comprising an agent effective for the treatment of hyperhidrosis and an assessment provided herein, or a portion thereof.

In some embodiments, the assessment is an ASDD, or a portion thereof. See Example 1. In some embodiments, the assessment is a version of the ASDD modified to measure hyperhidrosis in an area of the body other than the underarms (i.e., an SDD), or a portion of the SDD. Suitable other areas of the body include, for example, the palms of the hands, the soles of the feet, the face (including neck and scalp), the backs of the knees, the trunk, the groin, or other areas of the body.

In some embodiments, the assessment is a Weekly Impact Items questionnaire. See Example 2. In some embodiments, the assessment is a Weekly Impact Items questionnaire modified to measure hyperhidrosis in an area of the body other than the underarms. Suitable other areas of the body include, for example, the palms of the hands, the soles of the feet, the face (including neck and scalp), the backs of the knees, the trunk, the groin, or other areas of the body.

In some embodiments, the assessment is a Patient Global Impression of Change Item. See Example 3. In some embodiments, the assessment is a Patient Global Impression of Change Item modified to measure hyperhidrosis in an area of the body other than the underarms. Suitable other areas of the body include, for example, the palms of the hands, the soles of the feet, the face (including neck and scalp), the backs of the knees, the trunk, the groin, or other areas of the body.

In some embodiments, the pharmaceutical composition is provided in a form suitable for topical administration. In some aspects, the pharmaceutical composition is in the form of a pad or a wipe.

In some embodiments, the kit further comprises instructions for the administration of the pharmaceutical composition. In some aspects, the kit further comprises instructions for the administration of the assessment.

In some embodiments, the kit further comprises packaging. In some aspects, this packaging includes a container suitable for holding a pharmaceutical composition comprising a glycopyrronium compound and/or a further agent. The container can be made of any suitable material. Suitable materials include, for example, glass, plastic paper, laminates, and the like.

### 9. Patient Populations

In some embodiments, the methods of treatment provided herein are used to treat specific patient populations.

In some embodiments, the patient population is a patient population with a particular type of hyperhidrosis. In some aspects, the type of hyperhidrosis is selected from underarm hyperhidrosis, hyperhidrosis of the palms of the hands, hyperhidrosis of the soles of the feet, facial hyperhidrosis, neck hyperhidrosis, scalp hyperhidrosis, hyperhidrosis of the backs of the knees, trunk hyperhidrosis, groin hyperhidrosis, hyperhidrosis of other areas of the body, and combinations thereof.

In some embodiments, the patient population is a patient population of a particular age. In some aspects, the age of the patient population is selected from 5 to 14 years, 15 to 24 years, 25 to 34 years, 35 to 44 years, 45 to 54 years, 55 to 64 years, 65 to 74 years, 75 to 84 years, 85 to 94 years, 95 or more years, or combinations thereof.

In some embodiments, the patient population is a patient population of a particular gender. In some aspects, the gender of the patient population is female. In some aspects, the gender of the patient population is male. In some aspects, the male or female is a transgendered male or female.

In some embodiments, the patient population is a patient population with a family history of hyperhidrosis. In some aspects, the patient has an immediate family member (i.e., mother, father, brother, and/or sister) that suffers from hyperhidrosis. In some aspects, the patient has an extended family member (i.e., grandmother, grandfather, aunt, uncle, or cousin) that suffers from hyperhidrosis.

In some embodiments, the patient population is a patient population that has previously been treated, without success, by a method other than a method provided herein.

### 10. Combinations of Assessments Described Herein and Physiological Assessments

In some embodiments, the methods of treatment provided herein comprise administering one or more of the assessments described herein before, after, or concurrently with one or more physiological assessments. Physiological assessments may provide useful information that assists the healthcare practitioner in assessing the success in treating hyperhidrosis.

Any of the assessments provided herein may be used before, after, or concurrently with any suitable physiological assessment. In some aspects, the physiological assessment is administered before an assessment described herein. In some aspects, the physiological assessment is administered after an assessment described herein. In some aspects, the physiological assessment is administered concurrently with an assessment described herein.

In some aspects, “concurrently with” includes administration of a physiological assessment within a certain period of time before or after administration of an assessment provided herein. For example, in some aspects, a physiological assessment is considered to be administered concurrently with an assessment provided herein if the physiological assessment is administered within 1 hour, 2, hours, 3 hours, 4 hours, 8 hours, or 12 hours of an assessment provided herein.

In some aspects, a physiological assessment is administered before an assessment provided herein if it is administered more than 12 hours before the assessment provided herein. In some aspects, a physiological assessment is administered before an assessment provided herein if it is administered about 12 to about 24 hours before the assessment provided herein. In some aspects, a physiological assessment is administered before an assessment provided herein if it is administered about 1 to about 3 days before the assessment provided herein. In some aspects, a physiological assessment is administered before an assessment provided herein if it is administered about 3 to about 7 days before the assessment provided herein.

In some aspects, a physiological assessment is administered after an assessment provided herein if it is administered more than 12 hours after the assessment provided herein. In some aspects, a physiological assessment is administered after an assessment provided herein if it is administered about 12 to about 24 hours after the assessment provided herein. In some aspects, a physiological assessment is administered after an assessment provided herein if it is administered about 1 to about 3 days after the assessment provided herein. In some aspects, a physiological assessment is administered after an assessment provided herein if it is administered about 3 to about 7 days after the assessment provided herein.

In some aspects, the physiological assessment is selected from gravimetry (see e.g., Stefaniak et al., Clin. Auton. Res., 2013, 23:197-200, incorporated by reference in its entirety), vapometry (see Larson, Aesthet. Surg. J., 2011, 31:552-559, incorporated by reference in its entirety), electrical measurement of sweating (see Tronstad et al., Physiol. Meas., 2008, 29:S407-S415), visual measurement of sweating (see de Menezes Lyra, J. Bras. Pneumol., 2013, 39:521-522, incorporated by reference in its entirety), and combinations thereof.

### 11. Other Aspects of the Assessments Provided Herein

In some embodiments, the responses to the questions on one or more of the assessments provided herein, or portions thereof, may be aggregated using a scoring algorithm that creates a single score based on responses to multiple items on an assessment. The single score may useful to determine whether the treatment is successful overall when certain components of an assessment (e.g., severity of sweating over the past 24-hours) show improvement, but other components of the assessment (e.g., impact on activities) do not.

The specific scoring algorithm used to aggregate the scores may be selected by one of skill in the art. In some embodiments, numbers may be assigned to non-numeric responses to allow their aggregation into a single score. In some embodiments, the aggregation is performed by averaging (i.e., calculation of the mean or median). In some aspects, the average is a weighted mean that increases or decreases the influence of certain components on the weighted mean.

By way of illustration, in one embodiment, a numeric score is assigned to the answers of questions 1, 3, and 4 of the Axillary Sweating Daily Diary. See Example 1. The answer to question 2 of the Axillary Sweating Daily Diary is already in numeric form. For example, the answers to question 1 may be assigned to values of 1 (Yes) or 0 (No). Similarly, the answers to question 3 may be assigned to values of 0 (“Not at all”), 1 (“A little bit”), 2 (“A moderate amount”), 3 (“A great deal”), and 4 (“An extreme amount”). Finally, and similarly, the answers to question 4 may be assigned to values of 0 (“Not at all bothered”), 1 (“A little bothered”), 2 (“Moderately bothered”), 3 (“Very bothered”), and 4 (“Extremely bothered”).

A mean overall value for the four questions of the Axillary Sweating Daily Diary may then be calculated by averaging the numerical results for answers 1-4. Each component of the mean can be over- or under-weighted by applying a multiplier to reduce or increase the effect of each number on the overall average. If the multiplier is less than 1, the effect of a component will be underweighted. If the multiplier is greater than 1, the effect of the component will be overweighted. A similar effect can be achieved by using larger or smaller numbers to describe each component. The preceding numbering scheme is provided only for purposes of illustration. A skilled artisan can readily assign an appropriate numbering scheme and, if useful, weighting multiplier(s).

In some aspects, the responses to the Axillary Sweating Daily Diary (or corresponding version thereof, for other types of hyperhidrosis) are aggregated into a single score. In some aspects, the responses to the Weekly Impact Items (or corresponding version thereof, for other types of hyperhidrosis) are aggregated into a single score. In some aspects, the responses to the Axillary Sweating Daily Diary and the Weekly Impact Items (or corresponding versions thereof, for other types of hyperhidrosis) are aggregated into a single score. In some aspects, the responses to the Axillary Sweating Daily Diary, the Weekly Impact Items, and the Patient Global Impression of Change Item (or corresponding versions thereof, for other types of hyperhidrosis) are aggregated into a single score.

### EXAMPLES

### Example 1

### Axillary Sweating Daily Diary (ASDD)

The Axillary Sweating Daily Diary (ASDD) encompasses at least questions 1-4, below.

### Example 2

### Weekly Impact Items Questionnaire

The Weekly Impact Items Questionnaire encompasses at least questions a.-f., below.

### Example 3

### Patient Global Impression of Change Item

The Patient Global Impression of Change Item encompasses at least the question below.

### Patient Global Impression of Change Item

Overall, how would you rate your underarm sweating now as compared to before starting the study treatment?

1=Much better

2=Moderately better

3=A little better

4=No difference

5=A little worse

6=Moderately worse

7=Much worse

### Example 4

### Clinical Trials

Two randomized, double-blind, vehicle-controlled Phase 2 clinical trials, including a 198-patient, multi-center Phase 2b trial and a 38-patient Phase 2a trial, have demonstrated significant reductions in the signs and symptoms of primary axillary, or underarm, hyperhidrosis in patients treated with a topical formulation of the reference agent. The term “reference agent” refers to threo-glycopyrronium bromide. In these trials, patients treated with the topical formulation of the reference agent achieved significant improvements relative to baseline and relative to vehicle alone in widely used measures of hyperhidrosis severity.

### Example 4.1

### Phase 2a Clinical Trial

The completed Phase 2a clinical trial was a randomized, double-blind study in 38 patients with severe primary axillary hyperhidrosis. In six cohorts of six to seven patients each, two concentrations of the reference agent (2% and 4% (w/w)) in each of two topical formulations (A and B) were compared with their respective vehicles, which contain no active ingredient. These cohorts are summarized in Table 1.

TABLE 1
Cohorts for Phase 2a clinical trial.

| | Formulation A | | | Formulation B | | |
|---|---|---|---|---|---|---|
| | Concentration of reference agent | | | | | |
| | | | 0% | | | 0% |
| | 4% | 2% | (vehicle) | 4% | 2% | (vehicle) |
| Number of patients enrolled | 6 | 6 | 7 | 6 | 6 | 7 |

Patients were instructed to apply the study product once daily for four weeks using wipes saturated with either the topical formulation of the reference agent or vehicle only. Efficacy was evaluated based on axillary sweat production and disease severity. Assessments were conducted approximately weekly during the four-week treatment period and two weeks after the end of this treatment period. Disease severity was measured using a widely-used patient-reported outcome tool called the Hyperhidrosis Disease Severity Scale, or HDSS, wherein patients rate the severity of their disease on a four-point scale. Patients who rate the severity of their disease as a three or a four on the HDSS are considered to have severe disease, while those who rate it as one or two are considered to have mild or moderate disease. All 38 patients enrolled in the clinical trial rated the severity of their disease as a three or a four on the HDSS prior to the start of treatment. Trial inclusion criteria required that prior to the start of treatment, all patients produce at least 50 mg of sweat in each axilla over a five-minute period.

Overall, more patients in the groups treated with the topical formulations of the reference agent than in the vehicle-only groups experienced a reduction in axillary sweating and a reduction in disease severity over the four-week treatment period. Of the 38 patients enrolled in the study, 19 patients received one of the two formulations and 19 patients received the other formulation. Of the 19 who received the formulation selected for further development, referred to as Formulation A, all completed at least two weeks of treatment with the exception of one who received vehicle only. This patient withdrew due to an adverse event of dry mouth after one day of treatment with vehicle only and was excluded from the efficacy analysis.

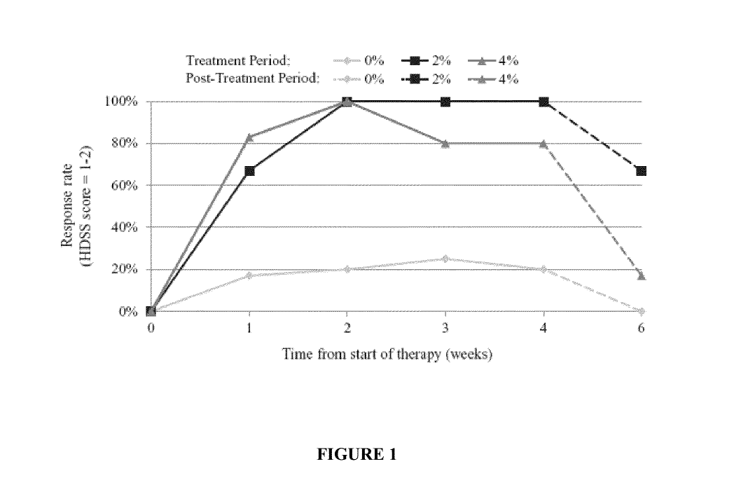

Presented in FIG. 1 and Table 2 are efficacy data for the 18 patients in whom the efficacy of Formulation A was assessed, including 12 who received the reference agent and six who received the vehicle only.

TABLE 2
Efficacy data for 18 patients in whom the efficacy of Formulation A was assessed.

| | | | | | | | | | | | Follow-up at | |
|---|---|---|---|---|---|---|---|---|---|---|---|---|
| | Pre- | | | | | | | | | | two weeks | |
| | treatment | | Treatment Period | | | | | | | | post-treatment | |
| Week | 0 | | 1 | | 2 | | 3 | | 4 | | 6 | |
| | Number of patients repoting at each time point | | | | | | | | | | | |
| 4% | 6 | | 6 | | 6 | | 5 | | 5 | | 6 | |
| 2% | 6 | | 6 | | 6 | | 5 | | 5 | | 6 | |
| Vehicle | 6 | | 6 | | 5 | | 4 | | 5 | | 6 | |
| Total | 18 | | 18 | | 17 | | 14 | | 15 | | 18 | |
| | Response rate (% of patients reporting mild or moderate disease severity at each time point) | | | | | | | | | | | |
| 4% | 0/6 | (0%) | 5/6 | (83%) | 6/6 | (100%) | 4/5 | (80%) | 4/5 | (80%) | 1/6 | (17%) |
| 2% | 0/6 | (0%) | 4/6 | (67%) | 6/6 | (100%) | 5/5 | (100%) | 5/5 | (100%) | 4/6 | (67%) |
| Vehicle | 0/6 | (0%) | 1/6 | (17%) | 1/5 | (20%) | 1/4 | (25%) | 1/5 | (20%) | 0/6 | (0%) |

Nine of 12 patients treated with the reference agent reported an improvement in their perception of disease severity, as assessed using the HDSS, from severe to mild or moderate at the first on-treatment assessment, which was conducted approximately one week after the start of therapy. By the second weekly assessment, all 12 severe hyperhidrosis patients treated with the reference agent reported their disease severity as mild or moderate, in comparison with one of six patients who received the vehicle only. In weeks three and four, five out of six patients in each of the 2% and 4% treatment groups reported on their disease severity. Of those patients who reported assessments, patients treated with the reference agent continued to report disease severity as mild or moderate in all reported assessments through the end of the four-week treatment period, except one patient who discontinued treatment after two weeks at the 4% dose who was therefore counted as a non-responder at weeks three and four.

Among the 14 patients treated with Formulation A whose sweat production was measured at the end of the four-week treatment period, sweat production in the nine patients treated with the reference agent was on average 61% lower than at the start of therapy, in comparison with an average 1% increase in sweat production in the five patients who received vehicle only. The effect of the reference agent on sweating and disease severity appeared to be reversible, as a trend toward a return to levels reported at the start of therapy was observed two weeks following cessation of therapy.

Adverse events observed across all 38 patients enrolled in the trial are summarized in Table 3. The proportions of patients enrolled in each cohort in which selected types of adverse events were reported are shown in parentheses.

TABLE 3
Adverse events observed across 38 patients.

| | Formulation A | | | Formulation B | | |
|---|---|---|---|---|---|---|
| | Concentration of reference agent | | | | | |
| | | | 0% | | | 0% |
| | 4% | 2% | (vehicle) | 4% | 2% | (vehicle) |
| Number of patients enrolled | 6 | 6 | 7 | 6 | 6 | 7 |
| Number of patients reporting | 4 (67%) | 4 (67%) | 5 (71%) | 5 (83%) | 3 (50%) | 4 (57%) |
| any adverse event (% of | | | | | | |
| patients enrolled) | | | | | | |
| Number of patients reporting | 2 (33%) | 3 (50%) | 5 (71%) | 3 (50%) | 3 (50%) | 3 (43%) |
| any treatment-related adverse | | | | | | |
| event (% of patients enrolled) | | | | | | |
| Number of treatment-related | 6 | 4 | 6 | 9 | 4 | 3 |
| adverse events | | | | | | |

Across all 38 patients enrolled in the trial, the most common treatment-emergent adverse events (TEAEs) were dry mouth and upper respiratory tract infection. No serious adverse events were reported. The occurrence of systemic adverse events appeared to be dependent on dose, regardless of the formulation used.

This Phase 2a clinical trial was designed to demonstrate proof-of-concept for the treatment of hyperhidrosis with the topical formulation of the reference agent and was not powered to demonstrate the statistical significance of any of the results.

### Example 4.2

### Phase 2b Clinical Program

Based on the Phase 2a data, a Phase 2b clinical program is being conducted in patients with primary axillary hyperhidrosis. The Phase 2b clinical program comprises two Phase 2b clinical trials:
Study DRM04-HH01, a dose-ranging study assessing the safety, efficacy and pharmacokinetics of the topical formulation of the reference agent in comparison with vehicle only in 198 patients, which was completed in August 2014; and
Study DRM04-HH02, an ongoing, dose-ranging study assessing the safety, efficacy and pharmacokinetics of DRM04, the topical formulation of the reference agent, and vehicle only in approximately 100 patients.

Both are randomized, multi-center, double-blind, vehicle-controlled trials in which the topical formulation of the reference agent, DRM04 or vehicle only, as applicable, is administered once daily for four weeks and efficacy is evaluated based on the HDSS and assessments of sweat production. The term “DRM04” refers to a topical formulation comprising threo-glycopyrronium tosylate. In addition to the HDSS, Study DRM04-HH02 uses a patient-reported outcome assessment provided herein we have developed (see Examples 1-3) that we believe may enable a more specific assessment of disease severity relative to the HDSS.

### Example 4.2.1

### Study DRM04-HH01

In Study DRM04-HH01, 198 patients with severe primary axillary hyperhidrosis were randomized to receive a topical formulation containing one of four concentrations of the reference agent (1%, 2%, 3% or 4%) or vehicle only. As in the Phase 2a clinical trial, patients were instructed to apply the study product once daily for four weeks using wipes saturated with either the topical formulation of the reference agent or vehicle only, and efficacy was evaluated based on axillary sweat production and the HDSS. Assessments were conducted approximately weekly during the four-week treatment period and the two-week period after the end of this treatment period. All 198 patients enrolled in the clinical trial rated the severity of their disease as a three or a four on the four-point HDSS prior to the start of treatment. Trial inclusion criteria required that prior to the start of treatment, all patients produce at least 50 mg of sweat in each axilla over a five-minute period.

The two primary efficacy endpoints evaluated in this trial were (1) the proportion of patients achieving an improvement of at least two points from baseline in HDSS score and (2) the average absolute change from baseline in sweat production, each as measured at the end of the four-week treatment period. For the purpose of the primary endpoint pertaining to sweat production, sweat production was assessed in each patient as the average of the amounts of sweat produced in each axilla during a five-minute period. As outlined below, the topical formulation of the reference agent demonstrated dose-dependent and, at certain doses, statistically significant improvements relative to vehicle in both primary efficacy endpoints.

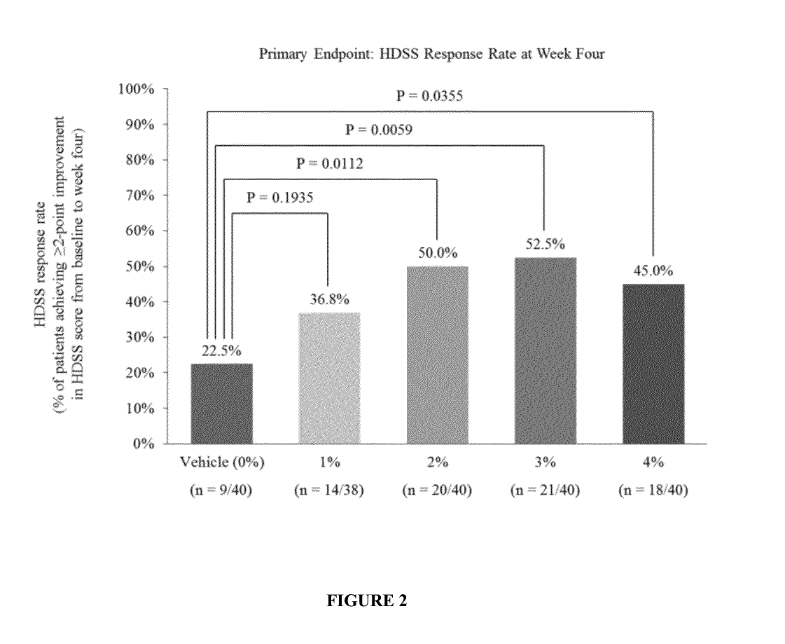

FIG. 2 summarizes the impact of the reference agent on disease severity, assessed as the proportion of patients achieving an improvement of at least two points in HDSS score from baseline to the end of the four-week treatment period. In FIG. 2, the p-values are an indication of statistical significance reflecting the probability of an observation occurring due to chance alone. A clinical trial result is statistically significant if it is unlikely to have occurred by chance. The statistical significance of clinical trial results is determined by a widely used statistical method that establishes the p-value of the results. Under this method, a p-value of 0.05 or less typically represents a statistically significant result.

At the end of the four-week treatment period, 36.8% to 52.5% of patients in each cohort treated with the reference agent achieved an improvement of at least two points in HDSS score, in comparison with 22.5% of patients who received the vehicle only. Based on these results, patients treated with the reference agent were between 63% and 133% more likely, depending on the concentration of the reference agent they received, to achieve an improvement of at least two points in HDSS score than patients who received the vehicle only.

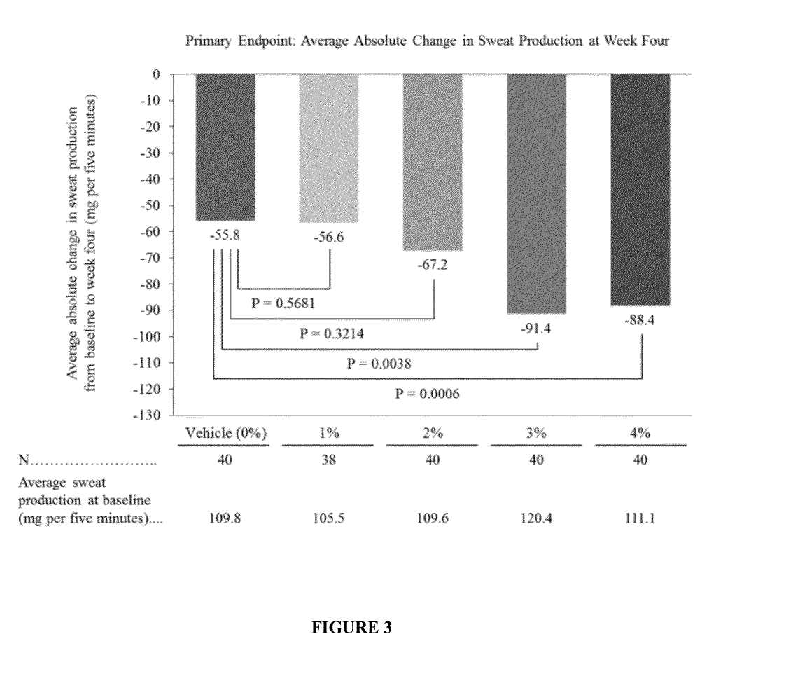

FIG. 3 summarizes the impact of the reference agent on sweat production, assessed as the average absolute change in sweat production from baseline to the end of the four-week treatment period. In FIG. 3, the p-values are an indication of statistical significance reflecting the probability of an observation occurring due to chance alone. A clinical trial result is statistically significant if it is unlikely to have occurred by chance. The statistical significance of clinical trial results is determined by a widely used statistical method that establishes the p-value of the results. Under this method, a p-value of 0.05 or less typically represents a statistically significant result.

From baseline to week four, patients in each cohort treated with the reference agent achieved an average reduction in sweat production of 56.6 to 91.4 mg, or 54.4% to 71.7%, in comparison with a reduction of 55.8 mg, or 43.2% in patients who received the vehicle only. The percentage reduction from baseline in sweat production was one of several non-primary efficacy analyses conducted in this trial.

In this trial, the most common TEAEs were dry mouth, upper respiratory tract infection, dry skin and blurred vision. Dry mouth, dry skin and blurred vision are well-known, reversible side effects of anticholinergic agents and were generally observed more frequently in patients who received higher concentrations of the reference agent. Upper respiratory tract infections were observed at similar frequencies in patients receiving the reference agent and patients receiving the vehicle only. Patients treated with the reference agent withdrew from the study due to adverse events rates of 2.6% in the 1% cohort (1/38), 5.0% in the 2% cohort (2/40), 2.5% (1/40) in the 3% cohort, and 20.0% (8/40) in the 4% cohort. None of the patients who received the vehicle only withdrew due to an adverse event. No treatment-related serious adverse events were reported.

### Example 4.2.2

### Study DRM04-HH02

Study DRM04-HH02 compared the topical formulation of the reference agent to DRM04. In this trial, approximately 100 patients with severe primary axillary hyperhidrosis were randomized into five cohorts of approximately 20 patients each. Each of the five cohorts was assigned to receive one of the following: a topical formulation containing one of two concentrations of the reference agent (i.e., threo-glycopyrronium bromide), DRM04 containing one of two concentrations of the novel form of the reference agent (i.e., threo-glycopyrronium tosylate), or vehicle only. The vehicle in the topical formulation of the reference agent was the same as in DRM04.

Patients were instructed to apply the study product once daily for four weeks using wipes saturated with the topical formulation of the reference agent, DRM04 or vehicle only. Efficacy was evaluated based on axillary sweat production, the HDSS and the patient-reported outcome assessment provided herein (see Examples 1-3) that we believe may enable a more specific assessment of disease severity relative to the HDSS. As in Study DRM04-HH01, assessments were scheduled approximately weekly during the four-week treatment period and the two-week period after the end of this treatment period. As in the Phase 2a clinical trial and Study DRM04-HH01, patients enrolled in Study DRM04-HH02 rated the severity of their disease as a three or a four on the HDSS and produce at least 50 mg of sweat in each axilla over a five-minute period prior to the start of treatment.

Results are provided in the tables below.

TABLE 4
Question 1

| | Reference 2.0% | Reference 3.0% | DRM04 2.5% | DRM04 3.75% | Vehicle |
|---|---|---|---|---|---|
| | (N = 21) | (N = 20) | (N = 22) | (N = 20) | (N = 22) |
| During Past 24 Hours (Quests 1-4) | | | | | |
| Question 1: Any underarm | | | | | |
| sweating? | | | | | |
| Baseline | | | | | |
| N | 20 | 20 | 21 | 20 | 21 |
| Mean (SD) | 1.00 (0.00) | 1.00 (0.02) | 0.98 (0.07) | 0.97 (0.08) | 1.00 (0.00) |
| Median | 1.00 | 1.00 | 1.00 | 1.00 | 1.00 |
| Minimum, Maximum | 1.0, 1.0 | 0.9, 1.0 | 0.7, 1.0 | 0.7, 1.0 | 1.0, 1.0 |
| Week 1 | | | | | |
| N | 21 | 20 | 22 | 20 | 22 |
| Mean (SD) | 0.90 (0.23) | 0.84 (0.27) | 0.84 (0.24) | 0.82 (0.30) | 1.00 (0.00) |
| Median | 1.00 | 1.00 | 1.00 | 1.00 | 1.00 |
| Minimum, Maximum | 0.3, 1.0 | 0.2, 1.0 | 0.1, 1.0 | 0.0, 1.0 | 1.0, 1.0 |
| Week 2 | | | | | |
| N | 21 | 20 | 22 | 20 | 22 |
| Mean (SD) | 0.79 (0.36) | 0.68 (0.43) | 0.75 (0.31) | 0.74 (0.37) | 0.99 (0.06) |
| Median | 1.00 | 1.00 | 0.90 | 1.00 | 1.00 |
| Minimum, Maximum | 0.0, 1.0 | 0.0, 1.0 | 0.0, 1.0 | 0.1, 1.0 | 0.7, 1.0 |
| Week 3 | | | | | |
| N | 21 | 20 | 22 | 20 | 22 |
| Mean (SD) | 0.68 (0.40) | 0.66 (0.45) | 0.75 (0.35) | 0.74 (0.38) | 0.95 (0.16) |
| Median | 0.80 | 1.00 | 1.00 | 1.00 | 1.00 |
| Minimum, Maximum | 0.0, 1.0 | 0.0, 1.0 | 0.0, 1.0 | 0.0, 1.0 | 0.3, 1.0 |
| Week 4 | | | | | |
| N | 21 | 20 | 22 | 20 | 22 |
| Mean (SD) | 0.64 (0.42) | 0.61 (0.44) | 0.73 (0.35) | 0.71 (0.38) | 0.95 (0.18) |
| Median | 0.80 | 0.80 | 0.95 | 1.00 | 1.00 |
| Minimum, Maximum | 0.0, 1.0 | 0.0, 1.0 | 0.0, 1.0 | 0.0, 1.0 | 0.2, 1.0 |
| Week 5 | | | | | |
| N | 19 | 18 | 20 | 20 | 19 |
| Mean (SD) | 0.68 (0.35) | 0.66 (0.41) | 0.85 (0.24) | 0.82 (0.34) | 0.91 (0.24) |
| Median | 0.80 | 0.90 | 1.00 | 1.00 | 1.00 |
| Minimum, Maximum | 0.0, 1.0 | 0.0, 1.0 | 0.2, 1.0 | 0.0, 1.0 | 0.2, 1.0 |
| Week 6 | | | | | |
| N | 19 | 19 | 20 | 20 | 19 |
| Mean (SD) | 0.79 (0.37) | 0.88 (0.27) | 0.89 (0.22) | 0.89 (0.31) | 0.93 (0.21) |
| Median | 1.00 | 1.00 | 1.00 | 1.00 | 1.00 |
| Minimum, Maximum | 0.0, 1.0 | 0.0, 1.0 | 0.3, 1.0 | 0.0, 1.0 | 0.3, 1.0 |

The baseline weekly average scores are not available for subjects initiated ASDD on their randomization date.

TABLE 5
Question 2

| | Reference 2.0% | Reference 3.0% | DRM04 2.5% | DRM04 3.75% | Vehicle |
|---|---|---|---|---|---|
| | (N = 21) | (N = 20) | (N = 22) | (N = 20) | (N = 22) |
| During Past 24 Hours (Quests 1-4) | | | | | |
| Question 2: Rate underarm | | | | | |
| sweating at its worst. | | | | | |
| Baseline | | | | | |
| N | 20 | 20 | 21 | 20 | 21 |
| Mean (SD) | 6.96 (1.90) | 6.95 (1.99) | 6.37 (2.22) | 6.48 (1.59) | 7.19 (1.67) |
| Median | 7.30 | 6.90 | 7.00 | 6.80 | 7.60 |
| Minimum, Maximum | 3.3, 10.0 | 3.0, 10.0 | 0.9, 9.0 | 2.5, 8.4 | 2.9, 9.8 |
| Week 1 | | | | | |
| N | 21 | 20 | 22 | 20 | 22 |
| Mean (SD) | 4.20 (2.37) | 4.23 (2.78) | 3.96 (2.26) | 4.14 (2.00) | 6.06 (2.13) |
| Median | 4.00 | 3.35 | 3.70 | 4.20 | 6.20 |
| Minimum, Maximum | 1.4, 8.8 | 0.5, 8.8 | 0.6, 7.9 | 0.0, 7.9 | 2.5, 9.7 |
| Absolute Change from Baseline at | | | | | |
| Week 1 | | | | | |
| N | 20 | 20 | 21 | 20 | 21 |
| Mean (SD) | −2.90 (2.66) | −2.73 (1.97) | −2.25 (1.94) | −2.33 (1.89) | −1.07 (1.44) |
| Median | −2.70 | −2.45 | −1.50 | −1.80 | −0.80 |
| Minimum, Maximum | −7.1, 2.1 | −6.3, 0.1 | −6.3, 0.6 | −5.8, 0.4 | −4.5, 0.9 |
| 95% Confidence Interval of Mean | −4.1, −1.7 | −3.6, −1.8 | −3.1, −1.4 | −3.2, −1.4 | −1.7, −0.4 |
| Week 2 | | | | | |
| N | 21 | 20 | 22 | 20 | 22 |
| Mean (SD) | 2.29 (1.83) | 2.58 (2.35) | 2.99 (2.36) | 2.92 (2.21) | 5.10 (2.37) |
| Median | 2.10 | 2.15 | 2.70 | 2.60 | 5.10 |
| Minimum, Maximum | 0.0, 6.3 | 0.0, 8.2 | 0.0, 7.9 | 0.1, 7.3 | 1.3, 10.0 |
| Absolute Change from Baseline at | | | | | |
| Week 2 | | | | | |
| N | 20 | 20 | 21 | 20 | 21 |
| Mean (SD) | −4.87 (2.57) | −4.37 (2.24) | −3.32 (2.37) | −3.56 (2.05) | −2.00 (1.77) |
| Median | −5.55 | −4.30 | −2.60 | −2.90 | −1.60 |
| Minimum, Maximum | −8.9, 0.7 | −8.0, −0.6 | −8.4, 0.6 | −7.3, −1.1 | −5.3, 0.3 |
| 95% Confidence Interval of Mean | −6.1, −3.7 | −5.4, −3.3 | −4.4, −2.2 | −4.5, −2.6 | −2.8, −1.2 |
| Week 3 | | | | | |
| N | 21 | 20 | 22 | 20 | 22 |
| Mean (SD) | 1.88 (1.80) | 2.33 (2.33) | 2.81 (2.17) | 2.77 (2.06) | 4.67 (2.56) |
| Median | 1.70 | 1.85 | 2.60 | 3.00 | 4.20 |
| Minimum, Maximum | 0.0, 6.0 | 0.0, 8.4 | 0.0, 7.6 | 0.0, 6.6 | 0.7, 9.6 |
| Absolute Change from Baseline at | | | | | |
| Week 3 | | | | | |
| N | 20 | 20 | 21 | 20 | 21 |
| Mean (SD) | −5.16 (2.68) | −4.62 (2.43) | −3.50 (2.35) | −3.71 (2.19) | −2.47 (2.39) |
| Median | −5.85 | −4.75 | −2.90 | −3.15 | −1.60 |
| Minimum, Maximum | −9.0, 0.4 | −8.9, −0.5 | −8.5, 0.3 | −7.8, −1.1 | −7.9, 0.6 |
| 95% Confidence Interval of Mean | −6.4, −3.9 | −5.8, −3.5 | −4.6, −2.4 | −4.7, −2.7 | −3.6, −1.4 |
| Week 4 | | | | | |
| N | 21 | 20 | 22 | 20 | 22 |
| Mean (SD) | 1.77 (1.75) | 2.05 (2.21) | 2.41 (2.10) | 2.48 (1.92) | 4.51 (2.73) |
| Median | 1.30 | 1.05 | 1.85 | 2.25 | 4.25 |
| Minimum, Maximum | 0.0, 6.0 | 0.0, 8.3 | 0.0, 7.3 | 0.0, 6.7 | 0.2, 9.7 |
| Absolute Change from Baseline at | | | | | |
| Week 4 | | | | | |
| N | 20 | 20 | 21 | 20 | 21 |
| Mean (SD) | −5.25 (2.83) | −4.91 (2.38) | −3.93 (2.48) | −4.00 (2.00) | −2.66 (2.37) |
| Median | −6.20 | −5.25 | −4.20 | −3.80 | −2.40 |
| Minimum, Maximum | −9.0, 0.6 | −9.1, −0.6 | −8.2, 0.2 | −7.7, −0.9 | −8.9, 0.7 |
| 95% Confidence Interval of Mean | −6.6, −3.9 | −6.0, −3.8 | −5.1, −2.8 | −4.9, −3.1 | −3.7, −1.6 |
| Week 5 | | | | | |
| N | 19 | 18 | 20 | 20 | 19 |
| Mean (SD) | 2.02 (1.84) | 1.56 (1.16) | 2.85 (2.13) | 2.82 (1.81) | 3.81 (2.25) |
| Median | 1.70 | 1.40 | 2.25 | 2.55 | 3.70 |
| Minimum, Maximum | 0.0, 7.2 | 0.0, 3.3 | 0.2, 7.5 | 0.0, 7.2 | 0.4, 7.6 |
| Absolute Change from Baseline at | | | | | |
| Week 5 | | | | | |
| N | 18 | 18 | 19 | 20 | 18 |
| Mean (SD) | −5.09 (2.56) | −5.29 (2.23) | −3.54 (2.46) | −3.66 (2.19) | −3.12 (2.63) |
| Median | −6.10 | −5.35 | −2.90 | −3.55 | −2.85 |
| Minimum, Maximum | −8.4, −0.4 | −9.2, −2.2 | −7.3, 0.4 | −7.7, −0.2 | −8.7, 0.8 |
| 95% Confidence Interval of Mean | −6.4, −3.8 | −6.4, −4.2 | −4.7, −2.4 | −4.7, −2.6 | −4.4, −1.8 |
| Week 6 | | | | | |
| N | 19 | 19 | 20 | 20 | 19 |
| Mean (SD) | 2.30 (1.75) | 3.02 (2.01) | 3.83 (2.20) | 3.44 (2.09) | 3.63 (2.33) |
| Median | 2.30 | 3.00 | 3.85 | 2.80 | 3.40 |
| Minimum, Maximum | 0.0, 7.4 | 0.0, 8.3 | 1.0, 8.0 | 0.0, 6.6 | 0.7, 8.2 |
| Absolute Change from Baseline at | | | | | |
| Week 6 | | | | | |
| N | 18 | 19 | 19 | 20 | 18 |
| Mean (SD) | −4.80 (2.46) | −3.83 (2.86) | −2.60 (1.85) | −3.04 (2.37) | −3.29 (2.61) |
| Median | −5.05 | −4.30 | −2.80 | −2.50 | −3.35 |
| Minimum, Maximum | −7.9, −0.5 | −9.0, 1.4 | −5.9, 0.5 | −7.4, 0.3 | −8.4, 0.5 |
| 95% Confidence Interval of Mean | −6.0, −3.6 | −5.2, −2.5 | −3.5, −1.7 | −4.1, −1.9 | −4.6, −2.0 |

The baseline weekly average scores are not available for subjects initiated ASDD on their randomization date.
Question 2 has a rating scale from 0 (No sweating at all) to 10 (Worst possible sweating).

TABLE 6
Question 3

| | Reference 2.0% | Reference 3.0% | DRM04 2.5% | DRM04 3.75% | Vehicle |
|---|---|---|---|---|---|
| | (N = 21) | (N = 20) | (N = 22) | (N = 20) | (N = 22) |
| During Past 24 Hours (Quests 1-4) [1] | | | | | |
| Question 3: How underarm sweating | | | | | |
| impact activities? | | | | | |
| Baseline | | | | | |
| N | 20 | 20 | 21 | 20 | 21 |
| Mean (SD) | 2.28 (0.80) | 2.12 (1.03) | 1.87 (1.01) | 2.11 (0.78) | 2.39 (0.76) |
| Median | 2.35 | 2.10 | 2.00 | 2.05 | 2.40 |
| Minimum, Maximum | 0.7, 4.0 | 0.6, 3.9 | 0.2, 3.4 | 0.7, 3.6 | 0.9, 3.6 |
| Week 1 | | | | | |
| N | 21 | 20 | 22 | 20 | 22 |
| Mean (SD) | 1.32 (0.82) | 1.24 (1.06) | 0.99 (0.79) | 1.40 (0.77) | 1.90 (0.93) |
| Median | 1.30 | 1.15 | 0.75 | 1.60 | 1.75 |
| Minimum, Maximum | 0.2, 3.2 | 0.1, 3.4 | 0.0, 2.9 | 0.3, 2.9 | 0.5, 3.8 |
| Absolute Change from Baseline at | | | | | |
| Week 1 | | | | | |
| N | 20 | 20 | 21 | 20 | 21 |
| Mean (SD) | −1.01 (1.05) | −0.89 (0.61) | −0.83 (0.81) | −0.72 (0.69) | −0.43 (0.60) |
| Median | −1.00 | −0.80 | −0.80 | −0.40 | −0.40 |
| Minimum, Maximum | −2.8, 1.1 | −2.0, 0.0 | −2.2, 0.6 | −2.3, 0.2 | −1.8, 0.4 |
| 95% Confidence Interval of Mean | −1.5, −0.5 | −1.2, −0.6 | −1.2, −0.5 | −1.0, −0.4 | −0.7, −0.2 |
| Week 2 | | | | | |
| N | 21 | 20 | 22 | 20 | 22 |
| Mean (SD) | 0.77 (0.66) | 0.79 (0.82) | 0.72 (0.79) | 0.94 (0.77) | 1.68 (0.87) |
| Median | 0.50 | 0.70 | 0.50 | 1.00 | 1.60 |
| Minimum, Maximum | 0.0, 2.0 | 0.0, 3.0 | 0.0, 2.9 | 0.0, 2.8 | 0.3, 4.0 |
| Absolute Change from Baseline at | | | | | |
| Week 2 | | | | | |
| N | 20 | 20 | 21 | 20 | 21 |
| Mean (SD) | −1.55 (1.02) | −1.34 (0.76) | −1.13 (0.94) | −1.17 (0.66) | −0.64 (0.69) |
| Median | −1.95 | −1.15 | −0.90 | −1.00 | −0.70 |
| Minimum, Maximum | −3.0, 0.3 | −2.9, 0.2 | −3.0, 0.6 | −2.5, 0.1 | −2.0, 0.6 |
| 95% Confidence Interval of Mean | −2.0, −1.1 | −1.7, −1.0 | −1.6, −0.7 | −1.5, −0.9 | −1.0, −0.3 |
| Week 3 | | | | | |
| N | 21 | 20 | 22 | 20 | 22 |
| Mean (SD) | 0.65 (0.68) | 0.65 (0.74) | 0.70 (0.73) | 0.85 (0.76) | 1.51 (0.99) |
| Median | 0.40 | 0.45 | 0.55 | 0.90 | 1.30 |
| Minimum, Maximum | 0.0, 2.0 | 0.0, 3.0 | 0.0, 2.6 | 0.0, 2.6 | 0.0, 4.0 |
| Absolute Change from Baseline at | | | | | |
| Week 3 | | | | | |
| N | 20 | 20 | 21 | 20 | 21 |
| Mean (SD) | −1.65 (1.08) | −1.48 (0.98) | −1.14 (0.91) | −1.26 (0.74) | −0.85 (0.89) |
| Median | −2.00 | −1.15 | −0.90 | −1.25 | −0.80 |
| Minimum, Maximum | −3.0, 0.5 | −3.5, −0.1 | −3.0, 0.3 | −2.6, 0.1 | −2.6, 0.6 |
| 95% Confidence Interval of Mean | −2.2, −1.1 | −1.9, −1.0 | −1.6, −0.7 | −1.6, −0.9 | −1.3, −0.4 |
| Week 4 | | | | | |
| N | 21 | 20 | 22 | 20 | 22 |
| Mean (SD) | 0.58 (0.62) | 0.53 (0.74) | 0.68 (0.69) | 0.71 (0.64) | 1.40 (1.07) |
| Median | 0.40 | 0.20 | 0.35 | 0.70 | 1.05 |
| Minimum, Maximum | 0.0, 1.9 | 0.0, 3.0 | 0.0, 2.3 | 0.0, 2.4 | 0.0, 4.0 |
| Absolute Change from Baseline at | | | | | |
| Week 4 | | | | | |
| N | 20 | 20 | 21 | 20 | 21 |
| Mean (SD) | −1.72 (1.06) | −1.60 (0.99) | −1.17 (0.89) | −1.40 (0.73) | −0.97 (0.97) |
| Median | −1.85 | −1.50 | −0.90 | −1.40 | −0.90 |
| Minimum, Maximum | −3.1, 0.3 | −3.7, −0.2 | −3.0, 0.0 | −2.7, −0.3 | −3.4, 0.6 |
| 95% Confidence Interval of Mean | −2.2, −1.2 | −2.1, −1.1 | −1.6, −0.8 | −1.7, −1.1 | −1.4, −0.5 |
| Week 5 | | | | | |
| N | 19 | 18 | 20 | 20 | 19 |
| Mean (SD) | 0.72 (0.63) | 0.46 (0.50) | 0.83 (0.76) | 0.91 (0.73) | 1.04 (0.76) |
| Median | 0.70 | 0.35 | 0.85 | 0.90 | 0.90 |
| Minimum, Maximum | 0.0, 2.0 | 0.0, 1.5 | 0.0, 2.7 | 0.0, 3.0 | 0.0, 2.5 |
| Absolute Change from Baseline at | | | | | |
| Week 5 | | | | | |
| N | 18 | 18 | 19 | 20 | 18 |
| Mean (SD) | −1.61 (0.99) | −1.59 (1.02) | −1.04 (0.89) | −1.20 (0.82) | −1.21 (1.02) |
| Median | −2.10 | −1.50 | −1.00 | −1.25 | −1.00 |
| Minimum, Maximum | −3.0, −0.2 | −3.7, 0.1 | −2.5, 0.4 | −2.7, 0.2 | −3.2, 0.2 |
| 95% Confidence Interval of Mean | −2.1, −1.1 | −2.1, −1.1 | −1.5, −0.6 | −1.6, −0.8 | −1.7, −0.7 |
| Week 6 | | | | | |
| N | 19 | 19 | 20 | 20 | 19 |
| Mean (SD) | 0.74 (0.60) | 0.88 (0.67) | 1.16 (0.86) | 1.22 (0.76) | 1.08 (0.80) |
| Median | 0.70 | 1.00 | 1.20 | 1.15 | 1.00 |
| Minimum, Maximum | 0.0, 1.8 | 0.0, 2.0 | 0.0, 2.6 | 0.0, 2.7 | 0.2, 3.0 |
| Absolute Change from Baseline at | | | | | |
| Week 6 | | | | | |
| N | 18 | 19 | 19 | 20 | 18 |
| Mean (SD) | −1.57 (0.98) | −1.16 (1.10) | −0.75 (0.59) | −0.89 (0.87) | −1.17 (1.08) |
| Median | −1.85 | −1.20 | −0.60 | −0.75 | −1.05 |
| Minimum, Maximum | −3.0, 0.1 | −3.0, 0.9 | −2.3, 0.2 | −2.7, 0.3 | −3.4, 0.6 |
| 95% Confidence Interval of Mean | −2.1, −1.1 | −1.7, −0.6 | −1.0, −0.5 | −1.3, −0.5 | −1.7, −0.6 |

The baseline weekly average scores are not available for subjects initiated ASDD on their randomization date.
Question 3 has a rating scale from 0 (Not at all) to 5 (An extreme amount).

TABLE 7
Question 4

| | Reference 2.0% | Reference 3.0% | DRM04 2.5% | DRM04 3.75% | Vehicle |
|---|---|---|---|---|---|
| | (N = 21) | (N = 20) | (N = 22) | (N = 20) | (N = 22) |
| During Past 24 Hours (Quests 1-4) | | | | | |
| Question 4: How bothered by | | | | | |
| underarm sweating? | | | | | |
| Baseline | | | | | |
| N | 20 | 20 | 21 | 20 | 21 |
| Mean (SD) | 2.45 (0.83) | 2.30 (1.07) | 2.03 (0.87) | 2.26 (0.79) | 2.51 (0.82) |
| Median | 2.55 | 2.10 | 2.00 | 2.30 | 2.40 |
| Minimum, Maximum | 0.9, 4.0 | 0.9, 4.0 | 0.3, 3.6 | 0.7, 3.9 | 0.6, 4.0 |
| Week 1 | | | | | |
| N | 21 | 20 | 22 | 20 | 22 |
| Mean (SD) | 1.40 (0.83) | 1.31 (1.09) | 1.07 (0.74) | 1.40 (0.84) | 2.06 (0.95) |
| Median | 1.30 | 1.25 | 0.85 | 1.60 | 1.95 |
| Minimum, Maximum | 0.0, 3.3 | 0.0, 3.4 | 0.0, 2.9 | 0.2, 3.0 | 0.6, 4.0 |
| Absolute Change from Baseline at | | | | | |
| Week 1 | | | | | |
| N | 20 | 20 | 21 | 20 | 21 |
| Mean (SD) | −1.10 (1.06) | −0.99 (0.58) | −0.90 (0.77) | −0.86 (0.65) | −0.42 (0.61) |
| Median | −1.00 | −0.85 | −0.80 | −0.65 | −0.40 |
| Minimum, Maximum | −2.7, 1.2 | −2.2, −0.3 | −2.3, 0.6 | −2.1, 0.1 | −1.8, 0.3 |
| 95% Confidence Interval of Mean | −1.6, −0.6 | −1.3, −0.7 | −1.3, −0.6 | −1.2, −0.6 | −0.7, −0.1 |
| Week 2 | | | | | |
| N | 21 | 20 | 22 | 20 | 22 |
| Mean (SD) | 0.78 (0.67) | 0.86 (0.84) | 0.84 (0.83) | 0.95 (0.82) | 1.79 (0.84) |
| Median | 0.60 | 1.00 | 0.65 | 0.90 | 1.70 |
| Minimum, Maximum | 0.0, 2.0 | 0.0, 3.2 | 0.0, 2.9 | 0.0, 2.8 | 0.6, 4.0 |
| Absolute Change from Baseline at | | | | | |
| Week 2 | | | | | |
| N | 20 | 20 | 21 | 20 | 21 |
| Mean (SD) | −1.70 (1.07) | −1.44 (0.74) | −1.17 (0.88) | −1.31 (0.70) | −0.70 (0.61) |
| Median | −1.80 | −1.10 | −1.00 | −1.35 | −0.70 |
| Minimum, Maximum | −3.3, 0.2 | −2.8, −0.3 | −3.0, 0.6 | −2.5, −0.1 | −2.0, 0.3 |
| 95% Confidence Interval of Mean | −2.2, −1.2 | −1.8, −1.1 | −1.6, −0.8 | −1.6, −1.0 | −1.0, −0.4 |
| Week 3 | | | | | |
| N | 21 | 20 | 22 | 20 | 22 |
| Mean (SD) | 0.71 (0.72) | 0.70 (0.79) | 0.87 (0.72) | 0.89 (0.79) | 1.65 (1.00) |
| Median | 0.40 | 0.50 | 0.85 | 1.00 | 1.70 |
| Minimum, Maximum | 0.0, 2.2 | 0.0, 3.2 | 0.0, 2.6 | 0.0, 2.6 | 0.0, 4.0 |
| Absolute Change from Baseline at | | | | | |
| Week 3 | | | | | |
| N | 20 | 20 | 21 | 20 | 21 |
| Mean (SD) | −1.76 (1.10) | −1.60 (0.98) | −1.15 (0.88) | −1.38 (0.75) | −0.85 (0.85) |
| Median | −1.75 | −1.30 | −1.10 | −1.35 | −0.70 |
| Minimum, Maximum | −3.3, 0.4 | −3.5, −0.5 | −2.5, 0.3 | −2.9, −0.3 | −2.6, 0.1 |
| 95% Confidence Interval of Mean | −2.3, −1.2 | −2.1, −1.1 | −1.6, −0.7 | −1.7, −1.0 | −1.2, −0.5 |
| Week 4 | | | | | |
| N | 21 | 20 | 22 | 20 | 22 |
| Mean (SD) | 0.66 (0.65) | 0.59 (0.75) | 0.75 (0.69) | 0.83 (0.72) | 1.55 (1.06) |
| Median | 0.70 | 0.45 | 0.75 | 1.00 | 1.30 |
| Minimum, Maximum | 0.0, 2.0 | 0.0, 3.1 | 0.0, 2.3 | 0.0, 2.4 | 0.1, 4.0 |
| Absolute Change from Baseline at | | | | | |
| Week 4 | | | | | |
| N | 20 | 20 | 21 | 20 | 21 |
| Mean (SD) | −1.81 (1.15) | −1.71 (1.03) | −1.28 (0.86) | −1.43 (0.79) | −0.97 (0.94) |
| Median | −1.95 | −1.35 | −1.20 | −1.35 | −1.00 |
| Minimum, Maximum | −3.3, 0.4 | −3.9, −0.3 | −2.9, 0.0 | −2.8, −0.2 | −3.5, 0.4 |
| 95% Confidence Interval of Mean | −2.3, −1.3 | −2.2, −1.2 | −1.7, −0.9 | −1.8, −1.1 | −1.4, −0.5 |
| Week 5 | | | | | |
| N | 19 | 18 | 20 | 20 | 19 |
| Mean (SD) | 0.77 (0.70) | 0.53 (0.48) | 0.98 (0.82) | 0.97 (0.74) | 1.21 (0.78) |
| Median | 0.90 | 0.50 | 1.00 | 1.00 | 1.00 |
| Minimum, Maximum | 0.0, 2.5 | 0.0, 1.3 | 0.0, 2.8 | 0.0, 3.0 | 0.1, 2.7 |
| Absolute Change from Baseline at | | | | | |
| Week 5 | | | | | |
| N | 18 | 18 | 19 | 20 | 18 |
| Mean (SD) | −1.74 (1.02) | −1.70 (1.09) | −1.11 (0.85) | −1.29 (0.84) | −1.17 (1.09) |
| Median | −1.95 | −1.85 | −1.00 | −1.25 | −1.10 |
| Minimum, Maximum | −3.1, −0.2 | −3.7, 0.1 | −2.7, 0.4 | −2.8, 0.2 | −3.5, 0.5 |
| 95% Confidence Interval of Mean | −2.2, −1.2 | −2.2, −1.2 | −1.5, −0.7 | −1.7, −0.9 | −1.7, −0.6 |
| Week 6 | | | | | |
| N | 19 | 19 | 20 | 20 | 19 |
| Mean (SD) | 0.93 (0.71) | 1.02 (0.55) | 1.19 (0.89) | 1.32 (0.75) | 1.21 (0.87) |
| Median | 1.00 | 1.00 | 1.05 | 1.30 | 1.00 |
| Minimum, Maximum | 0.0, 2.4 | 0.0, 2.0 | 0.2, 3.5 | 0.0, 2.7 | 0.0, 3.0 |
| Absolute Change from Baseline at | | | | | |
| Week 6 | | | | | |
| N | 18 | 19 | 19 | 20 | 18 |
| Mean (SD) | −1.56 (1.08) | −1.20 (1.24) | −0.90 (0.70) | −0.94 (0.95) | −1.17 (1.15) |
| Median | −1.90 | −1.30 | −0.90 | −0.80 | −1.15 |
| Minimum, Maximum | −3.0, 0.1 | −4.0, 1.0 | −2.2, 0.5 | −2.8, 0.2 | −3.5, 0.6 |
| 95% Confidence Interval of Mean | −2.1, −1.0 | −1.8, −0.6 | −1.2, −0.6 | −1.4, −0.5 | −1.7, −0.6 |

Question 4 has a rating scale from 0 (Not at all bothered) to 5 (Extremely bothered).

TABLE 8
Global Impression of Change

| | Reference 2.0% | Reference 3.0% | DRM04 2.5% | DRM04 3.75% | Vehicle |
|---|---|---|---|---|---|
| | (N = 21) | (N = 20) | (N = 22) | (N = 20) | (N = 22) |
| Overall | | | | | |
| Rate underarm sweating now as | | | | | |
| compared to before study | | | | | |
| Week 4 | | | | | |
| N | 18 | 14 | 15 | 17 | 17 |
| Mean (SD) | 1.39 (0.61) | 1.29 (0.73) | 1.40 (0.83) | 1.71 (0.99) | 2.35 (1.11) |
| Median | 1.00 | 1.00 | 1.00 | 1.00 | 2.00 |
| Minimum, Maximum | 1.0, 3.0 | 1.0, 3.0 | 1.0, 3.0 | 1.0, 4.0 | 1.0, 4.0 |
| 7: Much Worse | 0 | 0 | 0 | 0 | 0 |
| 6: Moderately Worse | 0 | 0 | 0 | 0 | 0 |
| 5: A Little Worse | 0 | 0 | 0 | 0 | 0 |
| 4: No Difference | 0 | 0 | 0 | 1 (5.0) | 3 (13.6) |
| 3: A Little Better | 1 (4.8) | 2 (10.0) | 3 (13.6) | 3 (15.0) | 5 (22.7) |
| 2: Moderately Better | 5 (23.8) | 0 | 0 | 3 (15.0) | 4 (18.2) |
| 1: Much Better | 12 (57.1) | 12 (60.0) | 12 (54.5) | 10 (50.0) | 5 (22.7) |
| Unknown | 3 (14.3) | 6 (30.0) | 7 (31.8) | 3 (15.0) | 5 (22.7) |
| Week 6 | | | | | |
| N | 14 | 9 | 14 | 12 | 13 |
| Mean (SD) | 2.07 (1.44) | 2.67 (1.12) | 2.50 (1.40) | 2.58 (1.31) | 2.31 (1.25) |
| Median | 1.50 | 3.00 | 3.00 | 3.00 | 2.00 |
| Minimum, Maximum | 1.0, 5.0 | 1.0, 5.0 | 1.0, 4.0 | 1.0, 4.0 | 1.0, 4.0 |
| 7: Much Worse | 0 | 0 | 0 | 0 | 0 |
| 6: Moderately Worse | 0 | 0 | 0 | 0 | 0 |
| 5: A Little Worse | 2 (9.5) | 1 (5.0) | 0 | 0 | 0 |
| 4: No Difference | 0 | 0 | 5 (22.7) | 4 (20.0) | 3 (13.6) |
| 3: A Little Better | 2 (9.5) | 4 (20.0) | 3 (13.6) | 3 (15.0) | 3 (13.6) |
| 2: Moderately Better | 3 (14.3) | 3 (15.0) | 0 | 1 (5.0) | 2 (9.1) |
| 1: Much Better | 7 (33.3) | 1 (5.0) | 6 (27.3) | 4 (20.0) | 5 (22.7) |
| Unknown | 7 (33.3) | 11 (55.0) | 8 (36.4) | 8 (40.0) | 9 (40.9) |

Question 6 is collected at Week 4 and Week 6 and is designed to reflect the change in underarm sweating experience as compared to before start of study treatment. This
question has a rating scale from 1 (Much better) to 7 (Much worse) with a midpoint 4 (No difference).

### 12. Equivalents

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

In particular, certain assessments provided herein use particular language (e.g., “a moderate amount”) or scales (e.g., 1-10, in increments of 1) in order to evaluate hyperhidrosis. Also included within the scope of the invention are methods that utilize assessments that are equivalent to (or obvious variants of) those provided for use with the methods provided herein. As a non-limiting example, a scale of A, B, C, D, E, F, G, H, I, and J, where A is best and J is worst, may be equivalent to (or an obvious variant of) a scale of 1, 2, 3, 4, 5, 6, 7, 8, 9, and 10, where 1 is best and 10 is worst. As another non-limiting example, the phrase “a medium amount” may be equivalent to (or an obvious variant of) the phrase “a moderate amount.” A person of skill in the art will readily recognize such equivalence or obvious variation.

1. A method for treating hyperhidrosis in a subject comprising:
a. scoring the subject's underarm sweating, during the past 24-hours, at its worst, on a scale of 0 to 10, at a value of 6 to 10 in a first scoring; and
b. administering an effective hyperhidrosis treatment to the subject such that after treatment, and at least 24-hours after the first scoring, the subject's underarm sweating is scored at a value of 0 to 5 in a second scoring.

2. The method of claim 1, further comprising continuing to administer the hyperhidrosis treatment to the subject, such that in a third scoring, at least 24-hours after the second scoring, the subject's underarm sweating is scored at a value of 0 to 5.

3. The method of claim 1, wherein the value in the first scoring is 7 to 10, and the value in the second scoring is 0 to 6.

4. The method of claim 1, further comprising adjusting the hyperhidrosis treatment administered to the subject based on the value in the second scoring.

5. The method of claim 2, wherein one or more of the first, second, and third scoring steps comprises administering a questionnaire to the subject.

6. The method of claim 5, wherein the questionnaire comprises a question asking the subject to rate their underarm sweating, during the past 24-hours, at its worst, on a scale of 0 to 10, in increments of 1.

7. A method for treating hyperhidrosis in a subject comprising:
a. scoring the impact of the subject's underarm sweating on their activities during the past 24-hours as “an extreme amount,” “a great deal,” or “a moderate amount” in a first scoring; and
b. administering an effective hyperhidrosis treatment to the subject such that after treatment, and at least 24-hours after the first scoring, the impact is “not at all” or “a little bit” in a second scoring.

8. The method of claim 7, further comprising continuing to administer the hyperhidrosis treatment to the subject, such that in a third scoring, at least 24-hours after the second scoring, the impact is “not at all” or “a little bit.”

9. The method of claim 7, wherein the impact in the first scoring is “an extreme amount” or “a great deal,” and the impact in the second scoring is “a moderate amount,” “a little bit,” or “not at all.”

10. The method of claim 7, further comprising adjusting the hyperhidrosis treatment administered to the subject based on the second scoring.

11. The method of claim 8, wherein one or more of the first, second, and third scoring steps comprises administering a questionnaire to the subject.

12. The method of claim 11, wherein the questionnaire comprises a question asking the subject to what extent their underarm sweating impacted their activities during the past 24-hours, on a scale of “not at all,” “a little bit,” “a moderate amount,” “a great deal,” and “an extreme amount.”

13. A method for treating hyperhidrosis in a subject comprising:
a. scoring how bothered the subject was by their underarm sweating, during the past 24-hours, as “extremely bothered,” “very bothered,” or “moderately bothered” in a first scoring; and
b. administering an effective hyperhidrosis treatment to the subject, such that after treatment, and at least 24-hours after the first scoring, the subject is “not at all bothered” or “a little bothered” in a second scoring.

14. The method of claim 13, further comprising continuing to administer the hyperhidrosis treatment to the subject, such that in a third scoring, at least 24-hours after the second scoring, the subject is “not at all bothered” or “a little bothered.”

15. The method of claim 13, wherein the subject is “extremely bothered” or “very bothered” in the first scoring and “moderately bothered,” “a little bothered,” or “not at all bothered” in the second scoring.

16. The method of claim 13, further comprising adjusting the hyperhidrosis treatment administered to the subject based on the second scoring.

17. The method of claim 14, wherein one or more of the first, second, and third scoring steps comprises administering a questionnaire to the subject.

18. The method of claim 17, wherein the questionnaire comprises a question asking the subject how bothered they were by their underarm sweating during the past 24-hours, on a scale of “not at all bothered,” “a little bothered,” “moderately bothered,” “very bothered,” and “extremely bothered.”

19. A method for treating hyperhidrosis in a subject comprising:
a. scoring whether the subject exhibited 6 symptoms of underarm hyperhidrosis during the past 7 days at a value of 4 to 6 symptoms exhibited out of the 6 symptoms in a first scoring; and
b. administering an effective hyperhidrosis treatment to the subject such that after treatment, and at least 7 days after the first scoring, the subject exhibits 0 to 3 symptoms in a second scoring.

20. The method of claim 19, further comprising continuing to administer the hyperhidrosis treatment to the subject, such that in a third scoring, at least 7 days after the second scoring, the subject exhibits 0 to 3 symptoms.

21. The method of claim 19, wherein the subject exhibits 5 to 6 symptoms in the first scoring and 0 to 4 symptoms in the second scoring.

22. The method of claim 19, further comprising adjusting the hyperhidrosis treatment based on the symptoms exhibited in the second scoring.

23. The method of claim 20, wherein one or more of the first, second and third scoring steps comprises administering a questionnaire to the subject.

24. The method of claim 23, wherein the questionnaire comprises a question asking the subject to respond with “yes” or “no” to the following questions:
a. “During the past 7 days, did you ever have to change your shirt during the day because of your underarm sweating?”;
b. “During the past 7 days, did you ever have to take more than 1 shower or bath a day because of your underarm sweating?”;
c. “During the past 7 days, did you ever feel less confident in yourself because of your underarm sweating?”;
d. “During the past 7 days, did you ever feel embarrassed by your underarm sweating?”;
e. “During the past 7 days, did you ever avoid interactions with other people because of your underarm sweating?”; and
f. “During the past 7 days, did your underarm sweating ever keep you from doing an activity you wanted or needed to do?”

25. A method for treating hyperhidrosis in a subject comprising:
a. administering an effective hyperhidrosis treatment to the subject; and
b. scoring the subject's overall underarm sweating at present, as compared to before treatment, as “much better,” “moderately better,” or “a little better” in a first scoring.

26. The method of claim 25, further comprising continuing to administer the hyperhidrosis treatment to the subject, such that in a second scoring, at least seven days after the first scoring, the subject's overall underarm sweating, as compared to before treatment, is scored as “much better,” “moderately better,” or “a little better.”

27. The method of claim 25, wherein the subject's overall underarm sweating in the first scoring is scored as “much better” or “moderately better.”

28. The method of claim 25, further comprising adjusting the hyperhidrosis treatment based on the first scoring.

29. The method of claim 26, wherein one or more of the first or second scoring steps comprises administering a questionnaire to the subject.

30. The method of claim 29, wherein the questionnaire comprises a question asking the subject to rate their underarm sweating now as compared to before starting the study treatment, on a scale of “much better,” “moderately better,” “a little better,” “no difference,” “a little worse,” “moderately worse,” and “much worse.”

31. The method of claim 5, wherein the questionnaire is selected from a written questionnaire, a verbal questionnaire, and an electronic questionnaire.

32. The method of claim 31, wherein the questionnaire is an electronic questionnaire, and a result from the electronic questionnaire is transmitted to a healthcare practitioner.

33. The method of claim 32, wherein the healthcare practitioner is selected from a physician, a nurse, and a pharmacist, and the result is used to adjust the hyperhidrosis treatment administered to the subject.

34. The method of claim 1, wherein the effective hyperhidrosis treatment is selected from an anticholinergic agent, a metal salt, a toxin, microwave heating, iontophoresis, surgical removal of sweat glands, sympathectomy, ultrasound, and a laser.

35. The method of claim 34, wherein the effective hyperhidrosis treatment is an anticholinergic agent.

36. The method of claim 35, wherein the anticholinergic agent is a glycopyrronium compound.

37. The method of claim 36, wherein the glycopyrronium compound is administered topically or orally.

38. The method of claim 37, wherein the glycopyrronium compound is administered topically, by a pharmaceutical composition comprising about 0.25% to about 6% (w/w) of the glycopyrronium compound.

39. The method of claim 38, wherein the glycopyrronium compound is selected from threo-glycopyrronium tosylate and threo-glycopyrronium bromide.

40. The method of claim 38, wherein the pharmaceutical composition is in the form of a pad or a wipe.

41. The method of claim 40, wherein the pad or the wipe further comprises ethanol.

42. The method of claim 40, wherein the pad or the wipe further comprises a hydrophilic polymer and a hydrophobic polymer.

43. The method of claim 42, wherein the hydrophilic polymer is polyvinyl pyrrolidone (PVP) and the hydrophobic polymer is a butyl ester of polyvinylmethylether/maleic anhydride (PVM/MA) copolymer.

44. The method of claim 40, wherein the pad or the wipe comprises a solution with a pH of about 3.5 to about 6.0.

45. The method of claim 1, wherein the subject's age is selected from 5 to 14 years, 15 to 24 years, 25 to 34 years, 35 to 44 years, 45 to 54 years, 55 to 64 years, 65 to 74 years, 75 to 84 years, 85 to 94 years, and 95 or more years.

46. The method of claim 1, wherein the subject has a family history of hyperhidrosis.

47. The method of claim 1, further comprising administering a physiological assessment of hyperhidrosis.

48. The method of claim 47, wherein the physiological assessment of hyperhidrosis is selected from gravimetry, vapometry, electrical measurement, visual measurement, and combinations thereof.

49. A kit comprising:
a. an agent effective for the treatment of hyperhidrosis; and
b. an assessment,
wherein the assessment comprises one or more of:
a Sweating Daily Diary, or a portion thereof;
a Weekly Impact Items questionnaire; and
a Patient Global Impression of Change Item.

50. The kit of claim 49, wherein the agent is a glycopyrronium compound.

51. The kit of claim 50, wherein the glycopyrronium compound is provided as a pharmaceutical composition comprising about 0.25% to about 6% (w/w) of the glycopyrronium compound.

52. The kit of claim 50, wherein the glycopyrronium compound is selected from threo-glycopyrronium tosylate and threo-glycopyrronium bromide.

53. The kit of claim 49, further comprising instructions for the administration of the assessment.

54. The kit of claim 49, further comprising instructions for the administration of the agent, based on the results of the assessment.
